(19) United States
(12) Patent Application Publication
(10) Pub. No.: US 2009/0176218 A1
(43) Pub. Date: Jul. 9, 2009

(54) Method for the Diagnosis and Prognosis of Cancer

(75) Inventors: Leo L. Cheng, Andover, MA (US); Ute Pohl, London (GB); Chin-Lee Wu, Newton, MA (US)

Correspondence Address: DAVID S. RESNICK, NIXON PEABODY LLP, 100 SUMMER STREET, BOSTON, MA, 02110-2131, US

(73) Assignee: THE GENERAL HOSPITAL CORPORATION, Boston, MA (US)

(21) Appl. No.: 12/090,005

(22) PCT Filed: Oct. 23, 2006
(86) PCT No.: PCT/US06/41655

§ 371 Date: Oct. 28, 2008

Related U.S. Application Data
(60) Provisional application No. 60/728,966, filed on Oct. 21, 2005.

(51) Int. Cl. C12Q 1/68

(52) U.S. Cl. 435/6

(57) ABSTRACT

Disclosed are methods for prognostic evaluation, and diagnosis of cancers. The methods of the present invention allow for the diagnosis and prognosis of cancers by analyzing the levels of expression of an enzyme that is involved in the biosynthetic pathway of spermine within tissue samples containing cancerous tissues. In particular, the amount of enzyme mRNA detected in tissue sample correlates with disease status such that enzyme levels can be used to predict the presence of, as well as the metastatic potential of cancer. Thus, measuring the level of spermine pathway enzymes in biological samples provides a quick, easy, and safe screen that can be used to both diagnose and prognose cancer in a patient thus leading to more effective treatments and cures.

### CROSS-REFERENCE TO RELATED APPLICATIONS

This application claims the benefit under 35 U.S.C. 119(e) of U.S. Provisional Patent Application Ser. No. 60/728,966, filed Oct. 21, 2005, the contents of which is herein incorporated by reference in their entirety.

### GOVERNMENT SUPPORT

This invention was supported by the National Institutes for Health (NIH) Grant No. RO1 CA095624 and DOD/US Army W81XWH-04-1-0190 and the Government of the United States has certain rights thereto.

### FIELD OF THE INVENTION

The present invention relates to methods for the diagnosis and prognosis of cancers by assessing levels of enzyme, such as Ornithine Decarboxylase (ODC) in a biological sample obtained form an individual.

### BACKGROUND

Cancers, for example, gastric cancer, prostate cancer, breast cancer, colon cancer, lung cancer, pancreatic cancer, ovarian cancer, cancer of the spleen, testicular cancer, cancer of the thymus, etc., are diseases characterized by abnormal, accelerated growth of epithelial cells. This accelerated growth initially causes a tumor to form. Eventually, metastasis to different organ sites can also occur. Although progress has been made in the diagnosis and treatment of various cancers, these diseases still result in significant mortality. While diagnostic methods have improved, methods to predict outcome have not. Thus, it is often the case that a diagnosis of cancer leads to confusion as to the appropriate treatment.

For example, prostate cancer is the most common cancer and second leading cause of cancer death among men in the United States. Heightened public awareness campaigns, the development of prostate specific antigen (PSA) tests, and the increased frequency of screening examinations have all contributed to a rising incidence in the diagnosis of early stage prostate cancers. These advancements have allowed prostate cancer patients to seek early treatment, and have improved the likelihood of successfully eradicating the disease. However, they have also generated confusion, as many cancers now detected might never be clinically symptomatic in the span of a normal life. A serious dilemma thus faces men and clinicians: if potentially lethal prostate cancers go undetected or are ignored, battles with life-threatening tumors may ensue. By contrast, the early detection of tumors that would have remained quiescent throughout life may lead to unnecessary treatments that may jeopardize the quality of a patient's remaining lifetime. Unfortunately, in the current prostate cancer clinic, no test can determine reliably which tumors pose so little threat that active treatment can, be deferred temporarily, or even indefinitely.

At present, a number of clinical tools are used to detect prostate cancer. Among them, the most widely applied methods are transrectal ultrasound and magnetic resonance imaging (spectroscopy), usually prescribed following patient symptoms, such as an abnormal digital rectal examination or a blood test indicating an elevated serum PSA level (3). When the results of these tests warrant additional exams, further investigations are often undertaken through the histopathological evaluation of tissue specimens obtained from an ultrasound-guided, transrectal needle biopsy. During prostate biopsies, in addition to sampling abnormal foci identified from imaging, traditional sextant tissue cores (three from the left and three from the right lobes of the prostate lobes), or more contemporary eight-site samples with emphasis on the peripheral zone, are removed with the biopsy gun. Pathologists are then given the opportunity to view the histopathological slides made from these specimens in order to grade the tumor (3).

Although several histological grading systems for prostate biopsy have been proposed for determining clinical potentials of prostate tumors (18-23), the Gleason Score is the most widely adopted system. This system assigns two “1 to 5” grades to primary (dominant) and secondary (sub-dominant) tumor growth patterns, respectively. Grade 1 represents very well differentiated tumors, while grade 5 shows no glandular differentiation. The sum of the two numbers determines tumor score, represented by a number between 2 to 10. Scores 2 to 4 are normally referred to as “well-differentiated disease”; 5 to 7 are named “moderately differentiated adenocarcinomas”; and 8 to 10 are called “poorly differentiated cancers.” (24) A higher score signifies greater probability of tumor extracapsular spread, nodal involvement, and metastases, according to current oncological understanding (25). More than 70% of cancers currently diagnosed belong to moderately differentiated adenocarcinomas, which the current histopathology cannot further sub-categorize. Additional clinical investigations are needed that often involve further surgical interventions, such as prostatectomies in order to assess whether or not the disease is organ-confined, evaluate the involvement of any pelvic lymph nodes, and seek distant metastases before determining the overall treatment strategy. The combination of clinical and histopathological findings generates another parameter, cancer stage, determined by the staging criteria of tumor, node, and metastasis (TNM) according to the American Joint Commission on Cancer (AJCC) (3, 7, 24).

Moderately Differentiated adenocarcinomase in prostate cancer present particular challenges to accurate diagnosis of the disease. Clinical experience routinely shows that tumor grades and stages are intrinsically related to some degree, especially when the result of a PSA test is also factored into consideration (26). Clinical decisions are relatively straight forward with regard to treatment for cases at either end of the grade and stage spectra; for cases where both parameters are very high or low, aggressive clinical intervention or regular monitoring of tumor development are respectively pursued. However, the majority of clinical cases observed are moderately differentiated tumors. A large body of clinical evidence shows that, for no obvious or known clinical reasons, the outcome for some patients is quite good, while for others it is poor (3, 7). Tumor aggressiveness manifested in biological activity is believed to be responsible for the variability of observed outcomes among individuals initially diagnosed with moderately differentiated prostate tumors. Presently no means exist in clinic to assess such bioactivities. Thus, modalities that can quantify such biological characteristics of tumor aggressiveness are urgently needed.

Spermine is a member of an aliphatic polyamine family that also includes putrescine and spermidine (54). Although more than 300 years have passed since it was first discovered in human semen, and despite the essential role that modern biology assigns to these cellular polyactionic polyamines in supporting eukaryotic cell growth, their exact molecular functions are still largely unknown. Studies of various functions of polyamines have been summarized in a recent review; they ranged from the basic biology of their counter-ionic effect in relation with the negative charges on RNA and DNA, to their utility as odorants in rodent social life (55). A number of medical hypotheses have also postulated spermine as an epidermal antioxidant (56) and a free radical scavenger (57). Despite the uncertainty over their biological functions, their metabolic pathway has been somewhat elucidated. It is now known that the biosynthesis of spermine starts with production of the diamine putrescine from ornithine catalyzed by the enzyme ornithine decarboxylase (ODC). Next, the addition of an aminopropyl group catalyzed by spermidine synthase (SpdS) converts putrescine to spermidine. Finally, addition of another aminopropyl group to spermidine in the presence of spermidine synthase (SpmS) generates spermine from spermidine (55). In this chain of biosynthetic reactions, ODC is believed to be a rate-limiting enzyme. However, overall cellular polyamine levels are believed to be regulated by multiple pathways, including synthesis, uptake, degradation and efflux (58-60).

### SUMMARY

The present invention is based on the surprising discovery that the mRNA level of ornithine decarboxylase (ODC) mRNA expression is upregulated in histologically-benign epithelia associated with progressive cancers. Accordingly, the present invention is directed to methods for prognostic evaluation, and diagnosis of cancers. The methods of the present invention allow for the diagnosis and prognosis of cancers by analyzing the levels of enzyme expression in tissue samples containing cancerous tissues. In particular, the amount of enzyme mRNA detected in tissue sample correlate with disease status such that enzyme levels can be used to predict the presence of, as well as the metastatic potential of cancer. Thus, measuring the level of spermine pathway enzymes in biological samples provides a quick, easy, and safe screen that can be used to both diagnose and prognose cancer in a patient thus leading to more effective treatments and cures.

The individual may also be screened levels in combination with other diagnostics such as, for example, additional biomarkers, mammography, manual examination, MRI, or tissue biopsy and histopathological examination.

The term “enzyme” is meant to include enzyme protein levels and/or enzyme mRNA levels throughout.

The term “normal control sample” refers to a biological sample obtained from a “normal” or “healthy” individual that does not have cancer. A normal control sample can also be a sample that contains the same concentration of ENZYME mRNA or protein normally present in a biological sample obtained from a healthy individual that does not have cancer.

The term “test sample” refers to a biological sample obtained from an individual suspected of having or at risk for developing cancer.

The term “biological samples”, is a tissue or tissue biopsy, e.g., a biopsy or core.

As used herein, “histopathologically-benign epithelia” means tissue that is morphologically normal according to the evaluation of a pathologist.

As used in the content of the present invention the term “enzyme” refers to enzymes in the spermine synthesis pathway, which include ornithine decarboxylase (ODC), spermidine synthase (SRM), spermine synthase (SMS), spermine oxidase (SMO), spermidine/spermine N-acetyltransferase (SSAT), and polyamine oxidase (PAO).

In one aspect of the present invention, the cancer is of epithelial origin. Examples of such cancers, which are encompassed in the present invention, are breast cancer, basal cell carcinoma, adenocarcinoma, gastrointestinal cancer, such as, for example, lip cancer, mouth cancer, esophageal cancer, small bowel cancer and stomach cancer, colon cancer, liver cancer, bladder cancer, pancreas cancer, ovary cancer, cervical cancer, lung cancer, and skin cancer, such as squamous cell and basal cell cancers, prostate cancer, renal cell carcinoma, and other known cancers that effect epithelial cells throughout the body. A preferred embodiment of the present invention is prostate cancer.

In another embodiment, the present invention provides a method for prognostic evaluation of a patient suspected of having, or having, a cancer. The method comprises the steps of i) measuring the level of spermine pathway enzyme mRNA present in histologically benign epithelia associated with the organ suspected of having, or having, cancer growth sample obtained from an individual, ii) to a level of enzyme known to be present in biological samples of the same type, which are obtained from healthy patients that do not have cancer, and evaluating the prognosis of the patient, where a high level of enzyme mRNA in the test sample indicates an aggressive form of cancer (e.g. metastatic or invasive) and therefore a poor prognosis. In one embodiment, the level of enzyme is compared to “histopathologically-benign epithelia”, which means tissue that is morphologically normal according to the evaluation of a pathologist.

In another embodiment, the level of enzyme mRNA is determined relative to a control, such as an endogenous control 18S rRNA, or “housekeeping” gene such as, for example, GAPDH or beta-actin. “Housekeeping” genes are known to those of skill in the art, see, for example, Trends in Genetics 19, 362-365 (2003), incorporated by reference in its entirety.

The present invention also contemplates the assessment of the level of enzyme present in multiple test samples obtained from the same patient, where a progressive increase in the amount of enzyme over time indicates an increased aggressiveness (e.g. metastatic potential) of the cancer.

In one aspect of the invention, enzyme levels present in a test biological sample are measured by analyzing the level of enzyme mRNA in a test sample and comparing this level to the level of enzyme in a control sample. In another embodiment, enzyme levels present in a test biological sample are measured by contacting the test sample, or preparation thereof, with an endogenous control, e.g., 18S rRNA. Laser capture microdissection (LCM) and RT-PCR can be used for the analysis of enzyme mRNA from tissue samples. Laser capture microdissection is known to those of skill in the art and described, for example, in Simon et al. (1998) Trends in Genetics 14:272 and Emmert-Buck et al. (1996) Science 274:998-1001.

In one embodiment, mRNA is isolated from the test sample and combined with reagents to convert it to cDNA. The converted cDNA is amplified and reagents and nucleic acid primers that hybridize to a nucleic acid molecule encoding enzyme are added to produce amplification products. These amplification products are analyzed so as to detect an amount of mRNA encoding enzyme. In one embodiment, mRNA levels from the test sample are compared to a panel of expected values for normal and malignant tissue derived using similar nucleic acid primers.

In another aspect of the invention, enzyme levels present in a test biological sample are measured by contacting the test sample, or preparation thereof, with an antibody-based binding moiety that specifically binds to enzyme protein, or to a portion thereof. The antibody-based binding moiety forms a complex with enzyme that can be detected, thereby allowing the levels of enzyme to be measured.

Any means known to those skilled in art can be used to assess enzyme levels. For example, in some embodiments enzyme expression levels are assayed by measuring levels of enzyme via mass spectrometry, ELISA, MR, CT, PET targeted at enzyme or immunohistochemistry.

In a further embodiment, the invention provides for kits that comprise means for measuring enzyme in a biological sample.

Other aspects of the invention are disclosed infra.

### DESCRIPTION OF THE FIGURES

The accompanying drawings, which are incorporated in and constitute a part of this specification, illustrate embodiments of the invention and, together with the description, serve to explain the objects, advantages, and principles of the invention.

FIG. 1: Eleven prostate cancer patients treated at MGH with whom multiple blood test results for prostate specific antigen (PSA) prior to prostatectomies were available, and from whom frozen tissue samples were selected for laser capture microdissection (LCM) of histologically benign glandular epithelia and followed by rt-PCR analysis of the LCM materials for ornithine decarboxylase (ODC) mRNA. Prostate tumor growth rates for these patients represented by the increase of PSA (PSA velocity, VPSA) were determined by linear regression analysis for each individual case as shown in this figure.

FIG. 2: See FIG. 1 with the addition of the ODC results obtained from LCM-rt-PCR. ODC results are presented as the fold of expression over the house-keeping gene 18S rRNA ([ODC]/[18S rRNA]). The “nd” denotes “Not Detectable” due to the lack of sufficient cellular materials of the sample from LCM.

FIG. 3: A plot of VPSA vs. [ODC]/[18S rRNA] for all nine cases with them ODC were measured.

FIG. 4: Same as FIG. 2, but with eliminations of cases based on the following two criteria: 1) ODC “nd” cases (P165 and P323); 2) the linearity of less than 85% (P203, P219, and P306). In addition, with the case P243, the clear outlier point (circled) was removed from the final calculation of VPSA. These eliminations resulted in five cases that are used for the final calculation of the relationship between VPSA and [ODC]/[18S rRNA].

FIG. 5: The final observed linear relationship between VPSA and [ODC]/[18S rRNA]. This plot indicates that by measuring [ODC]/[18S rRNA] at a single clinical point, the prostate tumor growth rate may be predicted and be utilized in clinical decision-making.

FIG. 6: Tests of RT-PCR conditions for ODC mRNA. 31 amplification cycles produce the best results. One Step RT-PCR was performed for up to 47 cycles. Tubes containing PCR reaction aliquots were taken out every 2 cycles starting at cycle 21. After quantification of the PCR products for each number of cycles, we determined that amplicon at cycle 31 was still in the linear range phase of the PCR amplification of ODC and thereafter, all reactions were performed for 31 cycles.

FIG. 7: Verification of ODC mRNA by three restriction enzymes (Nla IV, BstN1 and Nci I) that produce predicted fragments.

FIG. 8: Comparison between ODC mRNA obtained from total human prostate RNA of a commercial source (1) as a positive control and our own preparations from clinical tissues (2-6), negative control of water (7).

FIG. 9: Spectra and histopathology of (A) cancer and (B) normal samples obtained from the same prostate cancer patient. Chol, Choline; PCh, phosphorylcholine.

FIG. 10: Volume % of epithelial cells correlate with citrate and spermine measured from the same prostate specimens.

FIG. 11: Linear correlation between spermine and citrate measured from HRMAS 1HMRS.

FIG. 12: Pch+Chol differentiates Grade 6 (n=23) from Grade 7 (n=3) and 9 (n=4).

FIG. 13: Pch:Chol detects extraprostatic extension for Grade 6 tumors (Neg. n=15; Pos. n=2).

FIG. 14A-14C: 14A: HRMAS proton MRS indicates spermine; 14B: Quantitative pathology suggests the tissue used to obtain spectrum a) contains 40% normal epithelia, but no cancer cells; 14C: Equal amounts of epithelial (E) and stromal (S) tissues were laser dissected from the slide.

FIG. 15: Prostate epithelial tissue indicates much higher ODC activity than prostate stromal tissue of equal amount dissected from the same pathology slide.

FIG. 16: HRMAS spectra of human prostate tissue obtained with 400 Hz spinning rate on a 600 MHz spectrometer. 16A) with resonance suppression on tissue water, and 16B) without water suppression. Sidebands are indicated by arrows.

FIG. 17: HRMAS Spectra human prostate at spinning rate of 200, 300, 400, 1000 and 2000 Hz.

FIG. 18: The measured relationship between the intensity ratio of prostate metabolites (0.5-4.5 ppm) over the standard, and tissue weight at 400 Hz HRMAS rate.

FIG. 19: Examples of degradation curves measured, from intact prostate tissue, under HRMAS condition, at 3° C., for metabolites choline (Chol), spermine (Spm), creatine (Cr), and citrate (Citr).

FIG. 20: Comparisons of prostate metabolite degradation rates measured at 3° C. and 27° C.

FIG. 21: Comparisons of quantitative results obtained by visual estimation vs. computer-aided analysis.

FIG. 22: Serial sectioned histopathological images of human prostate tissue after HRMAS.

FIG. 23: An apparent quadratic approximation of relationship between the increasing PSA reading and spermine concentration.

### DETAILED DESCRIPTION

The present invention provides a novel method to diagnose and predict the growth rate of cancer in an individual affected with or at risk for developing cancer. In order to protect the biological sample from degradation prior to analysis, the biological sample is treated as to prevent degradation of enzyme protein, or enzyme mRNA. Methods for inhibiting or preventing degradation include, but are not limited to, treatment of the biological sample with protease or RNAase inhibitors, freezing the biological sample, or placing the biological sample on ice. Preferably, prior to analysis, the biological samples or isolates are constantly kept under conditions as to prevent degradation of enzyme protein, or enzyme RNA.

As used herein, a “tissue sample” refers to a portion, piece, part, segment, or fraction of a tissue which is obtained or removed from an intact tissue of a subject, preferably a human subject. One preferred tissue sample is prostate tissue.

As used herein, a “tumor sample” refers to a portion, piece, part, segment, or fraction of a tumor, for example, a tumor which is obtained or removed from a subject (e.g., removed or extracted from a tissue of a subject), preferably a human subject.

As used herein, a “primary tumor” is a tumor appearing at a first site within the subject and can be distinguished from a “metastatic tumor” which appears in the body of the subject at a remote site from the primary tumor.

As used herein, “ODC” refers to the ornithine decarboxylase. The term also encompasses species variants, homologues, allelic forms, mutant forms, and equivalents thereof.

Ornithine decarboxylase (ODC, EC 4.1.1.17) catalyzes conversion of L-ornithine to putrescine. Complete structure and nucleotide sequence of ornithine decarboxylase gene from mammalians is known for mouse (Coffino & Chen 1988, Katz & Kahana 1988), rat (van Steeg et al. 1988, Wen et al. 1989, van Steeg et al. 1990), human (Fitzgerald & Flanagan 1989, van Steeg et al. 1989, Hickok et al. 1990, Moshier et al. 1990), and bovine (Yao et al. 1998). In addition, the complete amino acid sequence (Srinivasan et al. 1987, Grens et al. 1989) and nucleotide sequence of the promoter region (GenBank accession number X53906) is known for hamster ODC. All mammalian ODC genes have 12 exons and 11 introns. Location of exon-intron boundaries is identical in all ODC genes. The transcription unit is relatively short, 6-8 kb depending on species. The first intron is considerably longer than others, 2.0-3.0 kb, and contains potential or demonstrated regulatory elements. Exons range from 85 bp to about 900 bp in length.

Mammals have several ODC gene-like sequences in their genomes, but apparently only one functional gene, others being pseudogenes. Active mouse ODC gene has been localized to chromosome 12 (Cox et al. 1988), functional hamster ODC gene is in the chromosome 7 (Tonin et al. 1987), human gene in the chromosome 2 (Winqvist et al. 1986, Hsieh et al. 1990, Radford et al. 1990) and rat gene in the chromosome 6 (Deng et al. 1994).

Northern analysis has revealed that rodents express two species of ODC mRNA (Berger et al. 1984, Kontula et al. 1984, Gilmour et al. 1985, van Kranen et al. 1987) whereas in human (Hickok et al. 1987, Radford et al. 1990) and bovine (Yao et al. 1995) only single ODC mRNA has been detected. The longer rodent mRNA is 2.6-2.7 kb in length, and the shorter 2.1-2.2 kb. They are the result of the use of two separate polyadenylation signals present at the 3′ untranslated region. Human ODC mRNA has two polyadenylation signals as well, but apparently only that giving the shorter transcript is used.

The open reading frame encoding mammalian ODC is 1383 nucleotides long the only exception being hamster ODC mRNA with the coding region of 1365 nucleotides. The coding region is highly conserved. Mouse and rat amino acid sequences differ only at 14 sites from each others. Hamster and bovine ODCs are the most divergent mammalian ODCs. They have 56 differences in amino acid sequences. The 5′-untranslated region of ODC mRNA is exceptionally long. Hamster has the ODC 5′ leader sequence slightly shorter than 300 nucleotides, those of other mammals exceed 300 nucleotides in length. The leader sequence is GC-rich and has been suggested to form secondary structures with a high free energy and stability (Brabant et al. 1988, Katz & Kahana 1988, Wen et al. 1989). The region also contains a small open reading frame as does AdoMetDC mRNA. This ORF may have a role in the regulation of translation, although the nucleotides flanking the translational start site do not conform perfectly well to the consensus sequence for the translation initiation (Kozak 1989).

ODC is one of the most highly regulated enzymes in eukaryotic organisms. The enzyme activity is induced rapidly up to several hundred fold by a great variety of factors stimulating cell growth and proliferation. These factors can be such as hormones, tumor promoters and growth factors. In almost all cases the increase in activity is accompanied by roughly equivalent changes in the amount of enzyme protein, and thus ODC appears not to be generally regulated by post-translational modifications or by allosteric effectors. The accumulation of ODC protein is controlled in gene transcription, mRNA translation and enzyme degradation. In addition, ODC activity is specifically inhibited by antizyme protein before degradation and negatively feed-back regulated by polyamines. (reviewed in (Davis et al. 1992, Shantz & Pegg 1999)

The present invention encompasses not only the detection of enzyme mRNA, but also enzyme protein.

The present invention is directed to methods for diagnosis and prognosis of cancer, in particular, cancers of epithelial origin in an individual. The methods involve measuring levels of enzyme in histopathologically benign epithelia associated with the organ having, or suspected of having, cancer growth, and in one embodiment, comparing the observed levels to levels of enzyme found in a normal control sample, for example a sample obtained from an individual that does not have cancer. Elevated levels of enzyme indicate the presence of progressive cancer. In one embodiment, ODC mRNA levels are measured.

To “compare” levels of enzyme expression means to detect gene expression levels in two samples and to determine whether the levels are equal or if one or the other is greater. A comparison can be done between quantified levels, allowing statistical comparison between the two values, or in the absence of quantification, for example using qualitative methods of detection such as visual assessment by a human.

As used herein, “a higher level (or elevated level) of enzyme in the test sample as compared to the level in the normal control sample” refers to an amount of enzyme that is greater than an amount of enzyme present in a normal control sample. The term “higher level” refers to a level that is statistically significant or significantly above levels found in the normal control sample.

The term “statistically significant” or “significantly” refers to statistical significance and generally means a two standard deviation (2SD) above normal, or higher, concentration of the marker.

For purposes of comparison, the test sample and normal control sample are of the same type, that is, obtained from the same biological source. The normal control sample can also be a standard sample that contains the same concentration of enzyme that is normally found in a biological sample of the same type and that is obtained from a healthy individual.

The present invention further provides for methods of prognostic evaluation of a patient suspected of having, or having, cancer, for example, a patient having an elevated PSA level. The method comprises measuring the level of enzyme (e.g., ODC mRNA) obtained from a patient in histologically benign epithelia associated with the organ having, or suspected of having, cancer growth. The levels may be measured at multiple time points. A high level, or level increasing over time, is indicative of a greater potential for metastatic activity and corresponds to a poor prognosis, while lower levels, or stable levels, indicate that the tumor is less aggressive and correspond to a better prognosis. The levels may be compared with the observed level with a range of enzyme levels normally found in biological samples (of the same type) of healthy individuals.

The prognostic methods of the invention also are useful for determining a proper course of treatment for a patient having cancer. A course of treatment refers to the therapeutic measures taken for a patient after diagnosis or after treatment for cancer. For example, a determination of the likelihood for cancer recurrence, spread, or patient survival, can assist in determining whether a more conservative or more radical approach to therapy should be taken, or whether treatment modalities should be combined. For example, when cancer recurrence or metastasis is likely, it can be advantageous to precede or follow surgical treatment with chemotherapy, radiation, immunotherapy, biological modifier therapy, gene therapy, vaccines, and the like, or adjust the span of time during which the patient is treated.

The levels of enzyme, as described herein, can be measured by any means known to those skilled in the art. In one embodiment of the present invention, the use of antibodies, or antibody equivalents, to detect levels of enzyme protein in biological samples is encompassed. However, other methods for detection of enzyme expression can also be used, such as measuring enzyme expression by analysis of mRNA transcripts. Measuring enzyme mRNA is preferred.

Methods for assessing levels of mRNA are well known to those skilled in the art. Preferred embodiments are herein described.

### Laser Capture Microdissection

Laser Capture Microdissection (LCM) is known to those of skill in the art, see, for example, Simon et al. (1998) Trends in Genetics 14:272 and Emmert-Buck et al. (1996) Science 274:998-1001. In one embodiment of the present invention a tumor sample or biopsy is obtained and LCM is used to obtain genetic material, such as, mRNA, for analysis.

Nucleic acid molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Rolff, A et al. PCR: Clinical Diagnostics and Research, Springer (1994).

Real time PCR is an amplification technique that can be used to determine levels of mRNA expression. (See, e.g., Gibson et al., Genome Research 6:995-1001, 1996; Heid et al., Genome Research 6:986-994, 1996). Real-time PCR evaluates the level of PCR product accumulation during amplification. This technique permits quantitative evaluation of mRNA levels in multiple samples. For mRNA levels, mRNA is extracted from a biological sample, e.g. a tumor and normal tissue, and cDNA is prepared using standard techniques. Real-time PCR can be performed, for example, using a Perkin Elmer/Applied Biosystems (Foster City, Calif.) 7700 Prism instrument. Matching primers and fluorescent probes can be designed for genes of interest using, for example, the primer express program provided by Perkin Elmer/Applied Biosystems (Foster City, Calif.). Optimal concentrations of primers and probes can be initially determined by those of ordinary skill in the art, and control (for example, beta-actin) primers and probes may be obtained commercially from, for example, Perkin Elmer/Applied Biosystems (Foster City, Calif.). To quantitate the amount of the specific nucleic acid of interest in a sample, a standard curve is generated using a control. Standard curves may be generated using the Ct values determined in the real-time PCR, which are related to the initial concentration of the nucleic acid of interest used in the assay. Standard dilutions ranging from 10-10⁶ copies of the gene of interest are generally sufficient. In addition, a standard curve is generated for the control sequence. This permits standardization of initial content of the nucleic acid of interest in a tissue sample to the amount of control for comparison purposes.

Methods of real-time quantitative PCR using TaqMan probes are well known in the art. Detailed protocols for real-time quantitative PCR are provided, for example, for RNA in: Gibson et al., 1996, A novel method for real time quantitative RT-PCR. Genome Res., 10:995-1001; and for DNA in: Heid et al., 1996, Real time quantitative PCR. Genome Res., 10:986-994.

A TaqMan-based assay also can be used to quantify MET polynucleotides. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, for example, AmpliTaq, results in the cleavage of the TaqMan probe. This cleavage separates the 5′ fluorescent dye and the 3′ quenching agent, thereby resulting in an increase in fluorescence as a function of amplification (see, for example, http://www2.perkin-elmer.com).

In another embodiment, for example, detection of RNA transcripts may be achieved by Northern blotting, wherein a preparation of RNA is run on a denaturing agarose gel, and transferred to a suitable support, such as activated cellulose, nitrocellulose or glass or nylon membranes. Labeled (e.g., radiolabeled) cDNA or RNA is then hybridized to the preparation, washed and analyzed by methods such as autoradiography.

Detection of RNA transcripts can further be accomplished using known amplification methods. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap lipase chain reaction (RT-AGLCR) as described by R. L. Marshall, et al., PCR Methods and Applications 4: 80-84 (1994). One suitable method for detecting enzyme mRNA transcripts is described in reference Pabic et. al. Hepatology, 37(5): 1056-1066, 2003, which is herein incorporated by reference in its entirety.

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; and target mediated amplification, as described by PCT Publication WO 9322461.

In situ hybridization visualization may also be employed, wherein a radioactively labeled antisense RNA probe is hybridized with a thin section of a biopsy sample, washed, cleaved with RNase and exposed to a sensitive emulsion for autoradiography. The samples may be stained with haematoxylin to demonstrate the histological composition of the sample, and dark field imaging with a suitable light filter shows the developed emulsion. Non-radioactive labels such as digoxigenin may also be used.

Alternatively, mRNA expression can be detected on a DNA array, chip or a microarray. Oligonucleotides corresponding to enzyme are immobilized on a chip which is then hybridized with labeled nucleic acids of a test sample obtained from a patient. Positive hybridization signal is obtained with the sample containing enzyme transcripts. Methods of preparing DNA arrays and their use are well known in the art. (See, for example U.S. Pat. Nos. 6,618,6796; 6,379,897; 6,664,377; 6,451,536; 548,257; U.S. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See for example U.S. Patent Application 20030215858).

To monitor mRNA levels, for example, mRNA is extracted from the biological sample to be tested, reverse transcribed, and fluorescent-labeled cDNA probes are generated. The microarrays capable of hybridizing to enzyme cDNA are then probed with the labeled cDNA probes, the slides scanned and fluorescence intensity measured. This intensity correlates with the hybridization intensity and expression levels.

### Quantitative PCR

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided, for example, in Innis et al. (1990) PCR Protocols, A Guide to Methods and Applications, Academic Press, Inc. N.Y.

Enzyme protein levels, or enzyme activity, can also be measured, in particular, when the biological sample is a fluid sample such as blood or urine. In one embodiment, levels of enzyme protein are measured by contacting the biological sample with an antibody-based binding moiety that specifically binds to enzyme, or to a fragment of enzyme. Formation of the antibody-enzyme complex is then detected as a measure of enzyme levels.

The term “antibody-based binding moiety” or “antibody” includes immunoglobulin molecules and immunologically active determinants of immunoglobulin molecules, e.g., molecules that contain an antigen binding site which specifically binds (immunoreacts with) to enzyme. The term “antibody-based binding moiety” is intended to include whole antibodies, e.g., of any isotype (IgG, IgA, IgM, IgE, etc), and includes fragments thereof which are also specifically reactive with enzyme protein. Antibodies can be fragmented using conventional techniques. Thus, the term includes segments of proteolytically-cleaved or recombinantly-prepared portions of an antibody molecule that are capable of selectively reacting with a certain protein. Non limiting examples of such proteolytic and/or recombinant fragments include Fab, F(ab′)2, Fab′, Fv, dAbs and single chain antibodies (scFv) containing a VL and VH domain joined by a peptide linker. The scFv's may be covalently or non-covalently linked to form antibodies having two or more binding sites. Thus, “antibody-base binding moiety” includes polyclonal, monoclonal, or other purified preparations of antibodies and recombinant antibodies. The term “antibody-base binding moiety” is further intended to include humanized antibodies, bispecific antibodies, and chimeric molecules having at least one antigen binding determinant derived from an antibody molecule. In a preferred embodiment, the antibody-based binding moiety detectably labeled.

“Labeled antibody”, as used herein, includes antibodies that are labeled by a detectable means and include, but are not limited to, antibodies that are enzymatically, radioactively, fluorescently, and chemiluminescently labeled. Antibodies can also be labeled with a detectable tag, such as c-Myc, HA, VSV-G, HSV, FLAG, V5, or HIS.

In the diagnostic and prognostic methods of the invention that use antibody based binding moieties for the detection of enzyme, the level of enzyme present in the biological samples correlate to the intensity of the signal emitted from the detectably labeled antibody.

In one preferred embodiment, the antibody-based binding moiety is detectably labeled by linking the antibody to an enzyme. The enzyme, in turn, when exposed to it's substrate, will react with the substrate in such a manner as to produce a chemical moiety which can be detected, for example, by spectrophotometric, fluorometric or by visual means. Enzymes which can be used to detectably label the antibodies of the present invention include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase.

Detection may also be accomplished using any of a variety of other immunoassays. For example, by radioactively labeling an antibody, it is possible to detect the antibody through the use of radioimmune assays. The radioactive isotope can be detected by such means as the use of a gamma counter or a scintillation counter or by audioradiography. Isotopes which are particularly useful for the purpose of the present invention are 3H, 131I, 35S, 14C, and preferably 125I.

It is also possible to label an antibody with a fluorescent compound. When the fluorescently labeled antibody is exposed to light of the proper wavelength, its presence can then be detected due to fluorescence. Among the most commonly used fluorescent labeling compounds are CYE dyes, fluorescein isothiocyanate, rhodamine, phycoerytherin, phycocyanin, allophycocyanin, o-phthaldehyde and fluorescamine.

An antibody can also be detectably labeled using fluorescence emitting metals such as 152Eu, or others of the lanthanide series. These metals can be attached to the antibody using such metal chelating groups as diethylenetriaminepentaacetic acid (DTPA) or ethylenediaminetetraacetic acid (EDTA).

An antibody also can be detectably labeled by coupling it to a chemiluminescent compound. The presence of the chemiluminescent-antibody is then determined by detecting the presence of luminescence that arises during the course of a chemical reaction. Examples of particularly useful chemiluminescent labeling compounds are luminol, luciferin, isoluminol, theromatic acridinium ester, imidazole, acridinium salt and oxalate ester.

As mentioned above, levels of enzyme protein can be detected by immunoassays, such as enzyme linked immunoabsorbent assay (ELISA), radioimmunoassay (RIA), Immunoradiometric assay (IRMA), Western blotting, immunocytochemistry or immunohistochemistry, each of which are described in more detail below. Immunoassays such as ELISA or RIA, which can be extremely rapid, are more generally preferred. Antibody arrays or protein chips can also be employed, see for example U.S. Patent Application Nos: 20030013208A1; 20020155493A1; 20030017515 and U.S. Pat. Nos. 6,329,209; 6,365,418, which are herein incorporated by reference in their entirety.

### Immunoassays

The most common enzyme immunoassay is the “Enzyme-Linked Immunosorbent Assay (ELISA).” ELISA is a technique for detecting and measuring the concentration of an antigen using a labeled (e.g. enzyme linked) form of the antibody. There are different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. 1984, J. Clin. Chem. Clin. Biochem., 22:895-904.

In a “sandwich ELISA”, an antibody (e.g. anti-enzyme) is linked to a solid phase (i.e. a microtiter plate) and exposed to a biological sample containing antigen (e.g. enzyme). The solid phase is then washed to remove unbound antigen. A labeled antibody (e.g. enzyme linked) is then bound to the bound-antigen (if present) forming an antibody-antigen-antibody sandwich. Examples of enzymes that can be linked to the antibody are alkaline phosphatase, horseradish peroxidase, luciferase, urease, and B-galactosidase. The enzyme linked antibody reacts with a substrate to generate a colored reaction product that can be measured.

In a “competitive ELISA”, antibody is incubated with a sample containing antigen (i.e. enzyme). The antigen-antibody mixture is then contacted with a solid phase (e.g. a microtiter plate) that is coated with antigen (i.e., enzyme). The more antigen present in the sample, the less free antibody that will be available to bind to the solid phase. A labeled (e.g., enzyme linked) secondary antibody is then added to the solid phase to determine the amount of primary antibody bound to the solid phase.

In a “immunohistochemistry assay” a section of tissue is tested for specific proteins by exposing the tissue to antibodies that are specific for the protein that is being assayed. The antibodies are then visualized by any of a number of methods to determine the presence and amount of the protein present. Examples of methods used to visualize antibodies are, for example, through enzymes linked to the antibodies (e.g., luciferase, alkaline phosphatase, horseradish peroxidase, or beta-galactosidase), or chemical methods (e.g., DAB/Substrate chromagen).

Alternatively, “Radioimmunoassays” may be employed. A radioimmunoassay is a technique for detecting and measuring the concentration of an antigen using a labeled (e.g. radioactively or fluorescently labeled) form of the antigen. Examples of radioactive labels for antigens include 3H, 14C, and 125I. The concentration of antigen enzyme in a biological sample is measured by having the antigen in the biological sample compete with the labeled (e.g. radioactively) antigen for binding to an antibody to the antigen. To ensure competitive binding between the labeled antigen and the unlabeled antigen, the labeled antigen is present in a concentration sufficient to saturate the binding sites of the antibody. The higher the concentration of antigen in the sample, the lower the concentration of labeled antigen that will bind to the antibody.

In a radioimmunoassay, to determine the concentration of labeled antigen bound to antibody, the antigen-antibody complex must be separated from the free antigen. One method for separating the antigen-antibody complex from the free antigen is by precipitating the antigen-antibody complex with an anti-isotype antiserum. Another method for separating the antigen-antibody complex from the free antigen is by precipitating the antigen-antibody complex with fornalin-killed S. aureus. Yet another method for separating the antigen-antibody complex from the free antigen is by performing a “solid-phase radioimmunoassay” where the antibody is linked (e.g., covalently) to Sepharose beads, polystyrene wells, polyvinylchloride wells, or microtiter wells. By comparing the concentration of labeled antigen bound to antibody to a standard curve based on samples having a known concentration of antigen, the concentration of antigen in the biological sample can be determined.

A “Immunoradiometric assay” (IRMA) is an immunoassay in which the antibody reagent is radioactively labeled. An IRMA requires the production of a multivalent antigen conjugate, by techniques such as conjugation to a protein e.g., rabbit serum albumin (RSA). The multivalent antigen conjugate must have at least 2 antigen residues per molecule and the antigen residues must be of sufficient distance apart to allow binding by at least two antibodies to the antigen. For example, in an IRMA the multivalent antigen conjugate can be attached to a solid surface such as a plastic sphere. Unlabeled “sample” antigen and antibody to antigen which is radioactively labeled are added to a test tube containing the multivalent antigen conjugate coated sphere. The antigen in the sample competes with the multivalent antigen conjugate for antigen antibody binding sites. After an appropriate incubation period, the unbound reactants are removed by washing and the amount of radioactivity on the solid phase is determined. The amount of bound radioactive antibody is inversely proportional to the concentration of antigen in the sample.

Other techniques may be used to detect enzyme, according to a practitioner's preference, based upon the present disclosure. One such technique is Western blotting (Towbin et al., Proc. Nat. Acad. Sci. 76:4350 (1979)), wherein a suitably treated sample is run on an SDS-PAGE gel before being transferred to a solid support, such as a nitrocellulose filter. Detectably labeled anti-enzyme antibodies can then be used to assess enzyme levels, where the intensity of the signal from the detectable label corresponds to the amount of enzyme present. Levels can be quantitated, for example by densitometry.

### Mass Spectometry

In addition, enzyme may be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference.

Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In certain embodiments, a gas phase ion spectrophotometer is used. In other embodiments, laser-desorption/ionization mass spectrometry is used to analyze the sample. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes. See, e.g., U.S. Pat. No. 5,118,937 (Hillenkamp et al.), and U.S. Pat. No. 5,045,694 (Beavis & Chait).

In SELDI, the substrate surface is modified so that it is an active participant in the desorption process. In one variant, the surface is derivatized with adsorbent and/or capture reagents that selectively bind the protein of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. In each of these methods, the derivatizing agent generally is localized to a specific location on the substrate surface where the sample is applied. See, e.g., U.S. Pat. No. 5,719,060 and WO 98/59361. The two methods can be combined by, for example, using a SELDI affinity surface to capture an analyte and adding matrix-containing liquid to the captured analyte to provide the energy absorbing material.

For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4.sup.th ed. Vol. 15 (John Wiley & Sons, New York 1995), pp. 1071-1094.

Detection of the presence of enzyme mRNA or protein will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of particular biomolecules. Software programs such as the Biomarker Wizard program (Ciphergen Biosystems, Inc., Fremont, Calif.) can be used to aid in analyzing mass spectra. The mass spectrometers and their techniques are well known to those of skill in the art.

In one preferred embodiment, enzyme levels are measured by MALDI-TOF mass spectrometry.

### Other Assays

Enzyme levels can also be measured by zymography, an assay well known to those skilled in the art and described in Heusen et al., Anal. Biochem., (1980) 102:196-202; Wilson et al., Journal of Urology, (1993) 149:653-658; Hernon al., J. Biol. Chem. (1986)261: 2814-2828, Braunhut et al., J. Biol. Chem. (1994) 269: 13472-13479; and Moses et al., Cancer Research 58, 1395-1399, Apr. 1, 1998, which are herein incorporated by reference in their entirety.

### Enzyme Antibodies

The antibodies for use in the present invention can be obtained from a commercial source. Alternatively, antibodies can be raised against enzyme polypeptide, or a portion of enzyme polypeptide. Methods for the production of enzyme antibodies are disclosed in PCT publication WO 97/40072 or U.S. Application. No. 2002/0182702, which are herein incorporated by reference.

Antibodies for use in the present invention can be produced using standard methods to produce antibodies, for example, by monoclonal antibody production (Campbell, A. M., Monoclonal Antibodies Technology: Laboratory Techniques in Biochemistry and Molecular Biology, Elsevier Science Publishers, Amsterdam, the Netherlands (1984); St. Groth et al., J. Immunology, (1990) 35: 1-21; and Kozbor et al., Immunology Today (1983) 4:72). Antibodies can also be readily obtained by using antigenic portions of the protein to screen an antibody library, such as a phage display library by methods well known in the art. For example, U.S. Pat. No. 5,702,892 (U.S.A. Health & Human Services) and WO 01/18058 (Novopharm Biotech Inc.) disclose bacteriophage display libraries and selection methods for producing antibody binding domain fragments.

### Enzyme Detection Kits

The present invention is also directed to commercial kits for the detection and prognostic evaluation of cancer. The kit includes a means for detecting enzyme levels in a biological sample such as antibodies, or antibody fragments, which selectively bind to enzyme protein, or a set of DNA oligonucleotide primers that allows synthesis of cDNA encoding the protein, or for example, a DNA probe that detects expression of enzyme mRNA. In one preferred embodiment, the kit comprises a means for detecting levels of enzyme in a sample of urine or blood.

Such kits may include any or all of the following: assay reagents, buffers, specific nucleic acids or antibodies (e.g. full-size monoclonal or polyclonal antibodies, single chain antibodies (e.g., scFv), or other gene product binding molecules), and other hybridization probes and/or primers, and/or substrates for polypeptide gene products.

In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### Method of Treating a Patient

In one embodiment, the invention provides a method for selecting a treatment program for a patient affected by or at risk for developing cancer by determining the presence or absence of enzyme. In this embodiment, an individual is tested for the presence or absence of enzyme and furthermore the levels of enzyme are compared against healthy control samples. If a diagnosis of cancer and poor prognosis are made, the patient should be treated aggressively for cancer. However, if a diagnosis and good prognosis are identified a more cautious, less invasive treatment program may be administered.

### DEFINITIONS

As used herein, “cancers of epithelial origin” refers to cancers that arise from epithelial cells which include, but are not limited to, breast cancer, basal cell carcinoma, adenocarcinoma, gastrointestinal cancer, lip cancer, mouth cancer, esophageal cancer, small bowel cancer and stomach cancer, colon cancer, liver cancer, bladder cancer, pancreas cancer, ovary cancer, cervical cancer, lung cancer, breast cancer and skin cancer, such as squamus cell and basal cell cancers, prostate cancer, renal cell carcinoma, and other known cancers that effect epithelial cells throughout the body.

The term “aggressive” or “invasive” with respect to cancer refers to the proclivity of a tumor for expanding beyond its boundaries into adjacent tissue (Darnell, J. (1990), Molecular Cell Biology, Third Ed., W. H. Freeman, NY). Invasive cancer can be contrasted with organ-confined cancer wherein the tumor is confined to a particular organ. The invasive property of a tumor is often accompanied by the elaboration of proteolytic enzymes, such as collagenases, that degrade matrix material and basement membrane material to enable the tumor to expand beyond the confines of the capsule, and beyond confines of the particular tissue in which that tumor is located.

The term “metastasis”, as used herein, refers to the condition of spread of cancer from the organ of origin to additional distal sites in the patient. The process of tumor metastasis is a multistage event involving local invasion and destruction of intercellular matrix, intravasation into blood vessels, lymphatics or other channels of transport, survival in the circulation, extravasation out of the vessels in the secondary site and growth in the new location (Fidler, et al., Adv. Cancer Res. 28, 149-250 (1978), Liotta, et al., Cancer Treatment Res. 40, 223-238 (1988), Nicolson, Biochim. Biophy. Acta 948, 175-224 (1988) and Zetter, N. Eng. J. Med. 322, 605-612 (1990)). Increased malignant cell motility has been associated with enhanced metastatic potential in animal as well as human tumors (Hosaka, et al., Gann 69, 273-276 (1978) and Haemmerlin, et al., Int. J. Cancer 27, 603-610 (1981)).

The present invention also encompasses the use of isolates of a biological sample in the methods of the invention. As used herein, an “isolate” of a biological sample (e.g., an isolate of a tissue or tumor sample) refers to a material or composition (e.g., a biological material or composition) which has been separated, derived, extracted, purified or isolated from the sample and preferably is substantially free of undesirable compositions and/or impurities or contaminants associated with the biological sample.

The term “antibody” is meant to be an immunoglobulin protein that is capable of binding an antigen. Antibody as used herein is meant to include antibody fragments, e.g. F(ab′)2, Fab′, Fab, capable of binding the antigen or antigenic fragment of interest. Preferably, the binding of the antibody to the antigen inhibits the activity of a variant form of EGFR.

The term “humanized antibody” is used herein to describe complete antibody molecules, i.e. composed of two complete light chains and two complete heavy chains, as well as antibodies consisting only of antibody fragments, e.g. Fab, Fab′, F (ab′)2, and Fv, wherein the CDRs are derived from a non-human source and the remaining portion of the Ig molecule or fragment thereof is derived from a human antibody, preferably produced from a nucleic acid sequence encoding a human antibody.

The terms “human antibody” and “humanized antibody” are used herein to describe an antibody of which all portions of the antibody molecule are derived from a nucleic acid sequence encoding a human antibody. Such human antibodies are most desirable for use in antibody therapies, as such antibodies would elicit little or no immune response in the human patient.

The term “chimeric antibody” is used herein to describe an antibody molecule as well as antibody fragments, as described above in the definition of the term “humanized antibody.” The term “chimeric antibody” encompasses humanized antibodies. Chimeric antibodies have at least one portion of a heavy or light chain amino acid sequence derived from a first mammalian species and another portion of the heavy or light chain amino acid sequence derived from a second, different mammalian species.

Preferably, the variable region is derived from a non-human mammalian species and the constant region is derived from a human species. Specifically, the chimeric antibody is preferably produced from a 9 nucleotide sequence from a non-human mammal encoding a variable region and a nucleotide sequence from a human encoding a constant region of an antibody.

A “cancer” in an animal refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an animal, or may be a non-tumorigenic cancer cell, such as a leukemia cell. In some circumstances, cancer cells will be in the form of a tumor; such cells may exist locally within an animal, or circulate in the blood stream as independent cells, for example, leukemic cells. Examples of cancer include but are not limited to breast cancer, a melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, colon cancer, colorectal cancer, esophageal cancer, gastrointestinal cancer, glioblastoma, hepatic carcinoma, hepatoma, kidney cancer, leukemia, liver cancer, lung cancer, lymphoma, non-small cell lung cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, urinary bladder cancer, uterine or endometrial cancer, and vulval cancer.

To “compare” levels of gene expression means to detect gene expression levels in two samples and to determine whether the levels are equal or if one or the other is greater. A comparison can be done between quantified levels, allowing statistical comparison between the two values, or in the absence of quantification, for example using qualitative methods of detection such as visual assessment by a human.

All references cited above or below are herein incorporated by reference.

The present invention is further illustrated by the following Examples. These Examples are provided to aid in the understanding of the invention and are not construed as a limitation thereof.

### EXAMPLES

The correlations between cellular metabolite gene expression and tumor progression described herein are expected to alleviate problems presently encountered in cancer diagnosis, particularly in the diagnosis of moderately differentiated tumors. The methods and results described herein and in the figures will allow for a clinician to obtain a tumor biological potential, as represented by the cellular metabolism of individual tumors, that will be of greater significance in directing treatment plans than the measured histopathological grade and stage used in today's clinic. Although these results are obtained with prostate cancer, it is expected that these applications are not limited to this particular form of cancer, but rather this type of diagnostic approach can be applied to a wide variety of cancers.

Analysis of Prostate Tumor Specimens using a Combined Approach of HRMAS 1HMRS, LCM, and RT-PCR

HRMAS 1HMRS (high resolution magic angle spinning proton magnetic resonance spectroscopy) was used to obtain accurate ex vivo measurements of individual metabolites without altering tissue pathological architectures (92-111). This method preserves tissue pathological structures for subsequent examination by histopathology and/or analysis by molecular biology, assisted by laser capture microdissection (LCM). In addition to this analysis, quantitative pathology and biomolecular analysis, aided by LCM, were performed on the same specimens. Using these techniques, prostate metabolites, localized in different prostatic pathological components, were analyzed, as were their relationship to tumor growth and progression, as clinically evaluated.

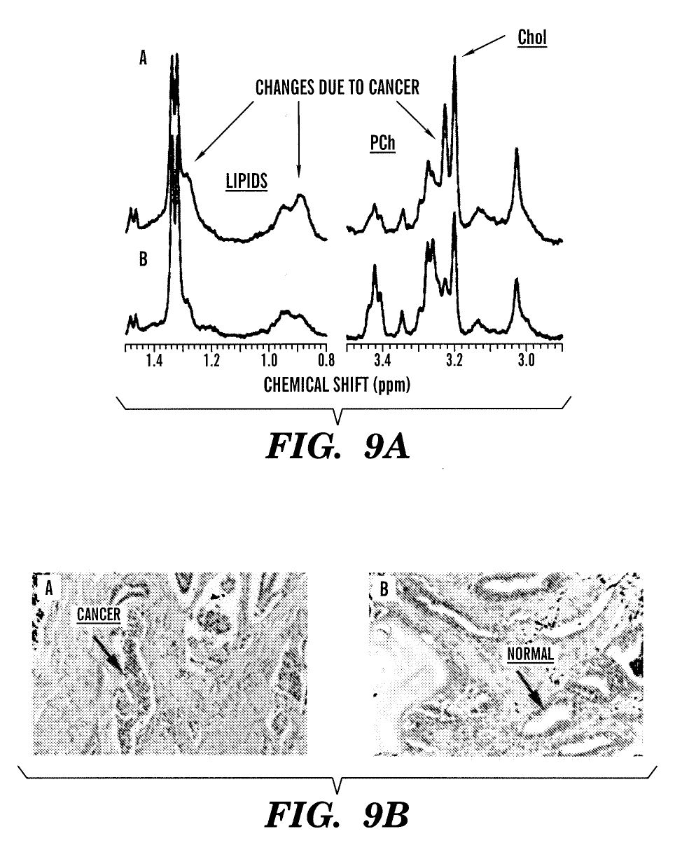

Due to the extreme heterogeneity of prostate cancer, a positive and complete diagnosis of cancer is currently achieved in clinical practice by multiple (>20) samplings through the entire, surgically removed prostate. FIG. 9 provides an example of the heterogeneity as seen with MRS (magnetic resonant spectroscopy) (top panel) and histopathology (bottom panels). This figure presents HRMAS 1HMRS and histopathology results obtained from two different tissue samples collected from the same patient (with Gleason Score 9/10). HRMAS 1HMRS was performed on these specimens to analyze the amounts of the cancer related metabolites (113, 114), phosphorylcholine (PCh), and lipids (115, 116). Phosphorylcholine and lipids were determined to be elevated in Spectrum A (cancerous tissue) as compared with Spectrum B (normal tissue). Histopathology conducted on these individual tissue samples after HRMAS 1HMRS revealed that cells in sample A (shown in the left, bottom panel) were mostly cancerous (60%) and stromal, while cells in sample B (shown in the right, bottom panel) were normal epithelia (17%) and stromal.

These results indicate the ability of HRMAS 1HMRS to detect cancer heterogeneity and its usefulness in differentiating cancerous from normal samples from an individual patient. It further emphasizes that, histopathological examination after MRS analysis, rather than reliance upon the clinical report, is critical to correctly interpret the spectroscopic results.

### Correlation Between Prostate Metabolites and Quantitative Pathology

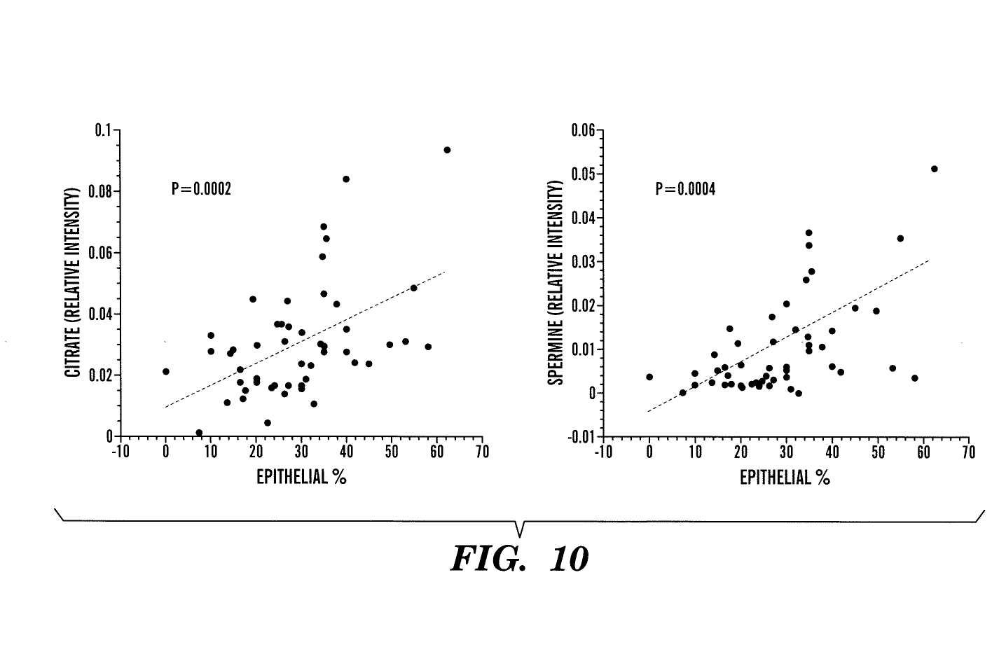

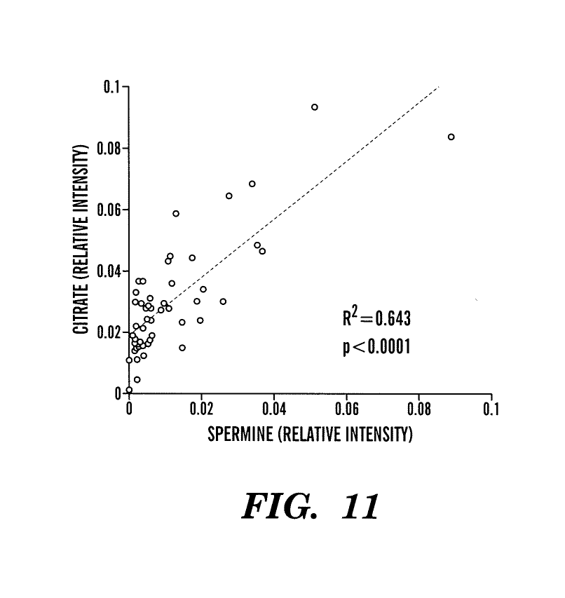

Human prostate surgical specimens for research were collected and incorporated (more than 60 surgical cases) into this study. From these specimens, more than 90 samples were measured with HRMAS 1HMRS. Quantitative pathology was performed on more than 60 samples from more than 50 cases. Previous correlations between the volume percentage of normal epithelial cells and the prostate metabolites spermine and citrate have been observed in studies which used 15 human prostate samples (97). The data presented in FIG. 10 was obtained using an extended study size. The results support the original observations. The data in FIG. 10 further indicates a higher statistical significance (p=0.0002 for citrate, and p=0.0004 for spermine) than those in the previous report (p=0.001 and 0.018, respectively) due to the current larger sample size. However, the r-square values have been reduced from 0.58 in the previous report to 0.25 for citrate, and from 0.36 to 0.23 for spermine. These reductions indicate that the current data sets are more widely scattered with respect to the linear relationship between prostate metabolites and the volume of normal epithelial prostate cells. Without being bound by theory, these observed deviations are thought to result from the linear relationship of the metabolites and tumor progression, and to reflect the intrinsic nature of the disease stage. Understanding and utilization of these metabolic markers requires molecular biological analysis of the relationship between prostate metabolites and each pathological feature. In addition, as previously reported, a strong linear correlation was observed between spermine and citrate (data shown in FIG. 11) which is in close agreement with results measured in human prostatic fluid (83).

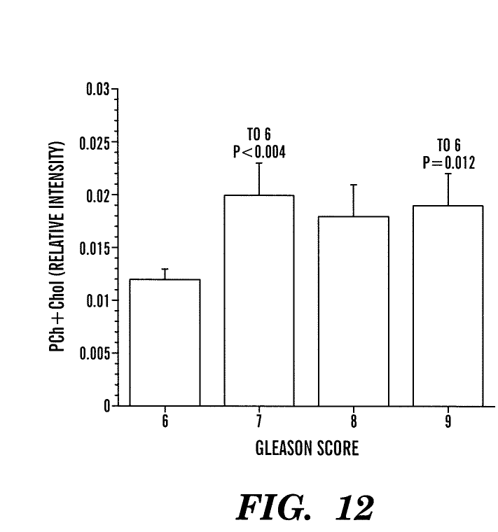

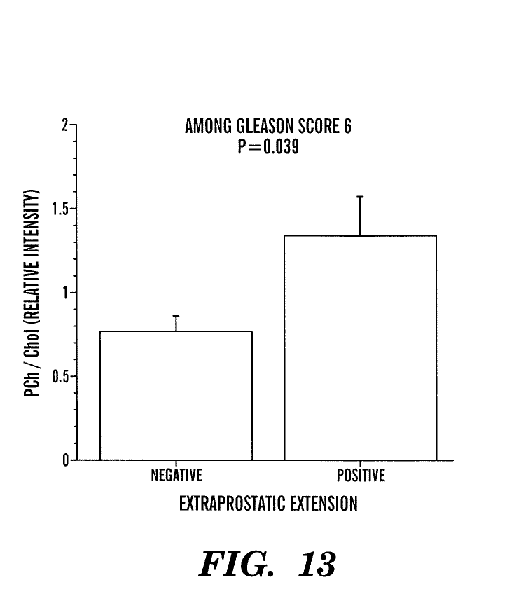

By combining the HRMAS 1HMRS results with the clinical data available for the prostate cancer cases studied, a number of metabolites were identified whose changes correlate with pathological evaluation and characterization. FIG. 12 shows that by using the increase in the sum of PCh and Chol (both are involved in cancer proliferation circles (113, 114)), the group of cancers with a Gleason Score of 6 (n=23) can be differentiated from those with Score 7 (n=3) and 9 (n=4) (statistical significance based on ANOVA analyses). Observation of the correlation between cholines and tumor clinical grades is not surprising; however, what was surprising was that the quantitative pathology of these samples indicated that, other than in all but one case of Score 9, no cancer cells were detected in any research specimen from a clinically proven cancer pathology. The quantitative pathology was performed through serial sections of entire tissue specimens. Cells in these specimens had the morphological appearance of normal epithelial cells; however, their cellular metabolites appeared to have been altered towards a direction that agrees with cancer development. Of additional note is that the relative changes between PCh and Chol may further differentiate the Score 6 group, which, known as moderately differentiated adenocarcinomas, represents the most challenging prostate cancer group in terms of current diagnosis (some of these patients will live with this disease throughout the course of a normal lifetime, while some will die of it without aggressive treatment). Our findings of PCh/Chol allowed us to divide Score 6 cases into two groups according to pathology status with respect to extraprostatic extension, an important prognostic parameter for prostate cancer, as shown in FIG. 13.

Correlations between metabolite concentrations measured with HRMAS 1HMRS and quantitative pathology, to identify metabolic signatures for normal prostate, BPH, PIN, and prostate adenocarcinomas were evaluated. To demonstrate the validity of these new markers and their utility in clinic, the first objective was to show their connection with existing knowledge of the disease, best represented by the pathological features characterized by the collective observations over the past century. Metabolic concentrations with HRMAS 1HMRS were also quantified for different histopathological components in different zones of normal human prostate. The HRMAS results have shown that metabolite changes can help differentiate between tumor types and establish correlations with tissue pathologies even without knowledge of normal metabolic levels in each prostate zone.

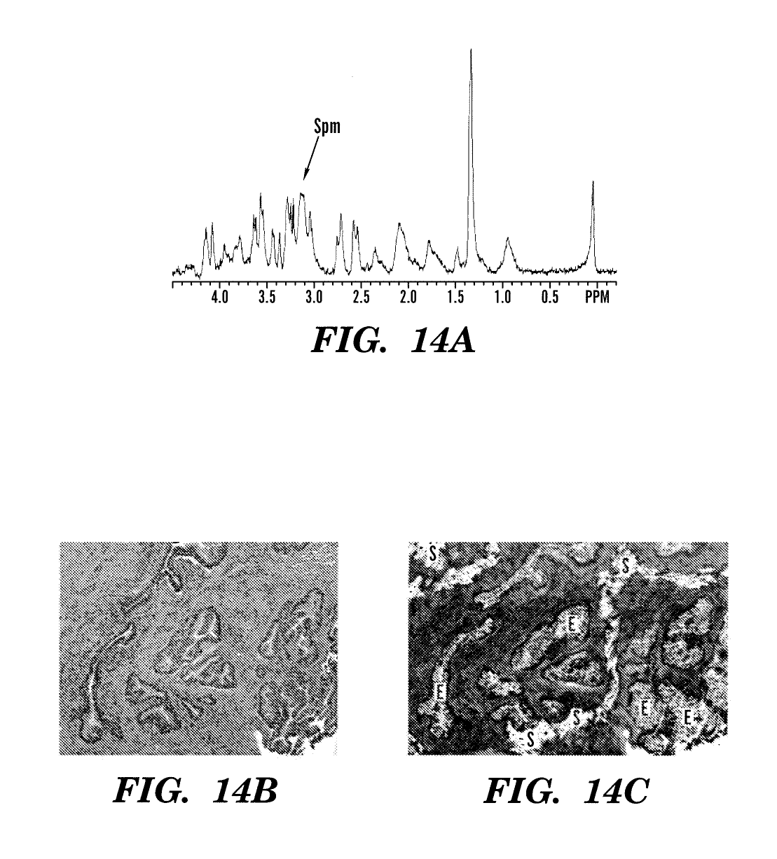

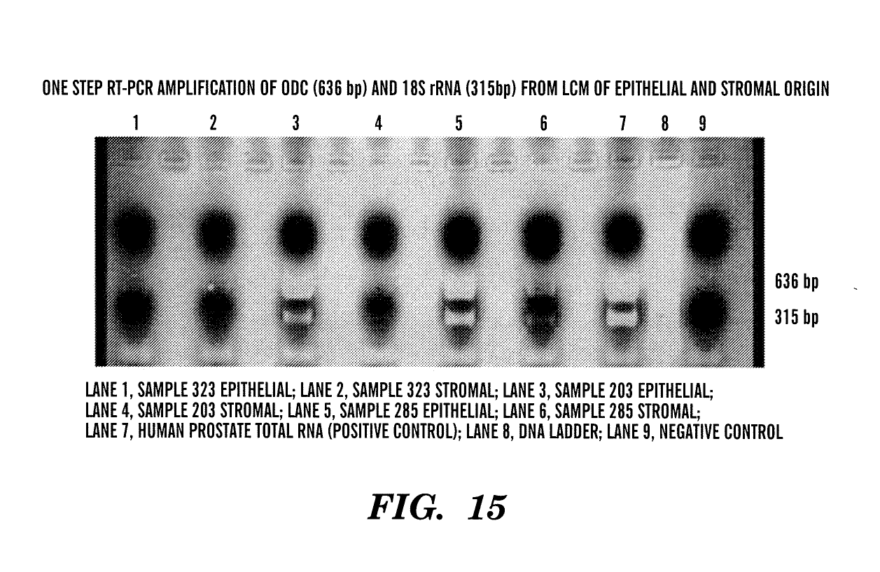

There have been previous reports of spermine functioning as an endogenous inhibitor of prostate cancer growth in cell line studies. Further, the amount of spermine in prostate tissue was found to linearly correlate with the amount of histologically benign epithelial cells (as discussed above). To test the proposed spermine inhibitory effect on prostate cancer growth with human cases, and to investigate the relationship between spermine production and prostate tumor progression, spermine in human prostate was studied at the molecular biological level. To do this, expression of the human genomic omithine decarboxylase (ODC), the key enzyme in the biosynthetic pathway of polyamines, resulting in spermine production (10, 46, 51-53, 55) was studied using HRMAS spectroscopy, quantitative pathology, LCM, and RT-PCR. FIG. 14A shows a tissue HRMAS spectrum obtained from a prostate cancer patient diagnosed with Gleason Score 6/10 (3/5+3/5). Prominent spermine resonances were observable in the spectrum. After the spectroscopy study, the sample was frozen and cut into 7 μm pathology slides by using a cryostat. Quantitative pathology revealed that the studied specimen consisted of 40% epithelial cells, as shown in FIG. 14B, but no cancer cells were detected. FIG. 14C shows the adjacent pathology image, from which equal amounts of prostatic epithelial and stromal tissue had been lifted by LCM according to the NIH procedures (http://cgap-mf.nih.gov). Laser-dissected epithelial and stromal materials were processed separately and simultaneously according to the standard NIH protocols established for micro RNA separation and purification after LCM (http://cgap-mf.nih.gov). The purified epithelial and stromal RNA samples were then subjected to RT-PCR amplification using primers specific for ODC (121) mRNA (636 bp) and 18S rRNA (315 bp), where 18S rRNA was used as an endogenous control. The sizes of the amplified ODC and 18S rRNA were verified by gel electrophoresis, as shown in FIG. 15.

The results from these experiments indicate that: 1) it is possible to laser-dissect different prostate pathology features for molecular biology analysis from the same tissue sample after HRMAS spectroscopy; and 2) ODC is concentrated in the epithelial cells rather than universally distributed over all prostate cells. Since ODC is the rate-limiting enzyme for spermidine production, this result strongly supports the above reported correlation between spermine expression and epithelial cell type.

### Statistical Evaluating of the Relationship Between Spermine and PSA Velocity.

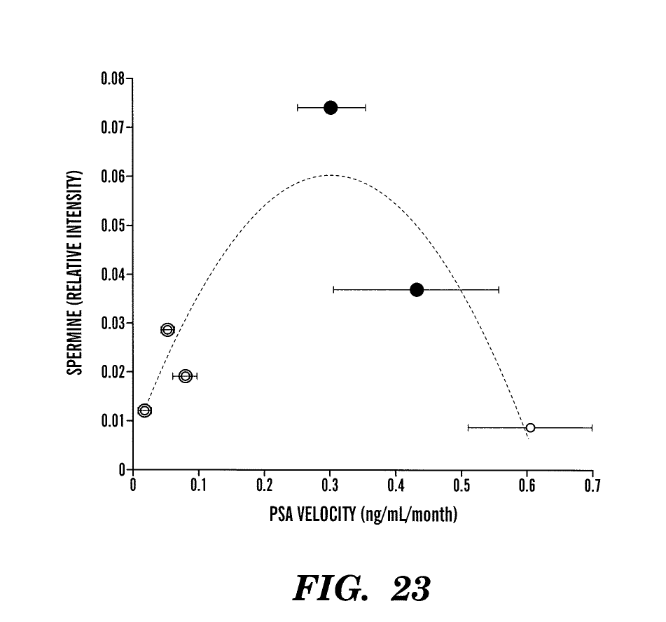

The relationship between the spermine concentration in normal epithelial cells of prostate surgical specimens and the increase in patient PSA readings (PSA velocity, a indicator for tumor growth rate (4)) six months to two years prior to the surgeries was preliminarily investigated for six cases with available clinical data. An indicated quadratic approximation with the maximum centered at the PSA velocity of 0.3 ng/ml/month (R2=0.81), is shown in FIG. 23. This relationship is in agreement with the hypothesis that the concentration of spermine in epithelial cells increases in order to inhibit tumor growth in the early stages, but then decreases after a certain point when tumor growth becomes too rapid to maintain a normal rate of enzymatic spermine production.

RT-PCR Amplification of the Messenger RNA(mRNA) of ODC from Laser-Dissected Prostate Materials.

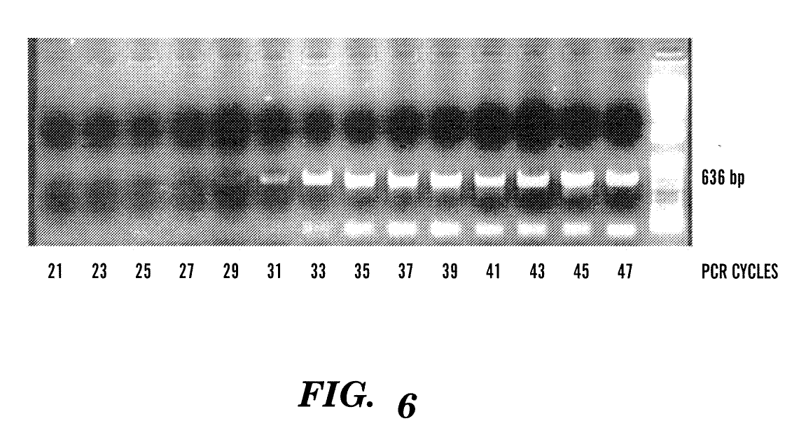

In order to further investigate the relationship between spermine biosynthesis and tumor growth, expression of the rate limiting enzyme in spermine production, ODC, was analyzed in prostate tumor. The experimental steps involved were systematically designed and evaluated as follows. First, the oligonucleotide primers reported in the literature (121) on a commercially available total human RNA sample (CLONTECH) were tested. These tests included variation of RT-PCR conditions (annealing temperature, PCR cycles, etc) to optimize the amplification of the ODC sequence. The optimal RT-PCR conditions for the 636-nucleotide ODC sequence isolated by the two primers (121) was determined to be: a one-step cDNA synthesis and denaturation at 50° C. for 30 min followed by 94° C. for 2 min; 31 amplification cycles consisting of 30 sec at 94° C. (denaturation); 30 sec at 60° C. (annealing); and 45 sec at 72° C. (primer extension) and for 10 min at 72° C. final extension (see FIG. 6). To ensure that the observed 636-nt band was the result of ODC amplification of the target and not amplification of RNA introduced from an external source, a negative control was prepared by using only RNase. The absence of a band for the negative controls indicated that experimental conditions were sufficiently sterile to prevent the contamination from an outside source (such as the RNA present in the air or in the laboratory working area).

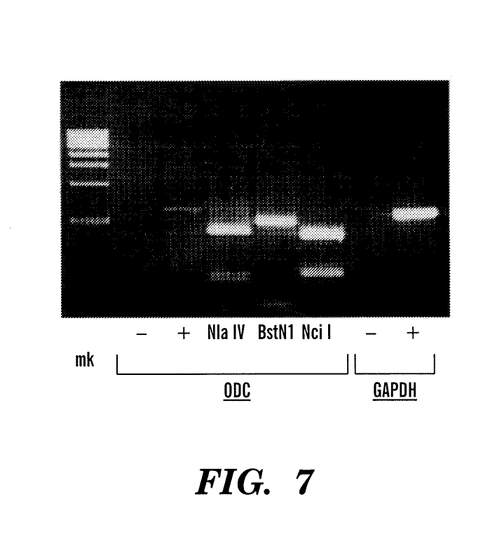

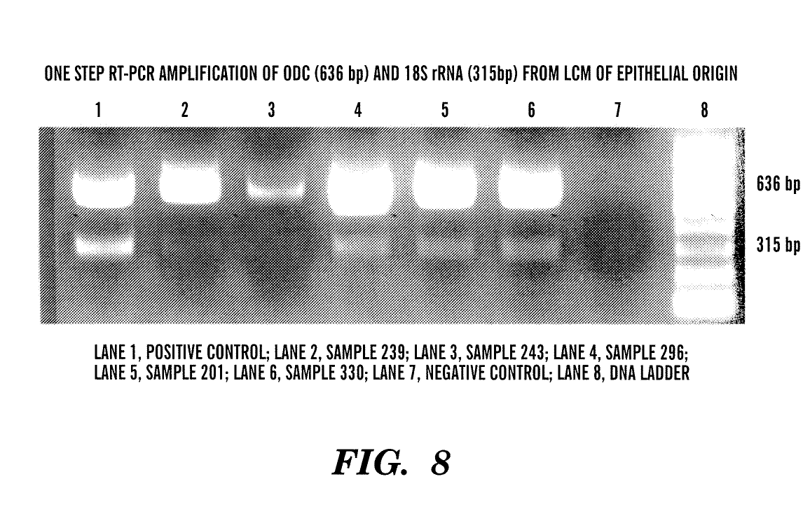

Next, the RT-PCR product was verified as the targeted ODC gene by cutting the product individually with three restriction enzymes at unique sites within the amplified region. The cutting products were expected to generate distinct fragments. Digest with Nla IV was predicted to generate 451 and 185 nt fragments, digest with BstN1 was expected to generate 539 and 97 nt fragments, and digest with Nci I was expected to generate 431 and 205 nt fragments. The restriction enzyme digests verified that the expected sequence was recognized by the primers, as shown in FIG. 7. In the next step, human prostate RNA was isolated from research samples according to the procedure of Biogene FastPrep RNA Isolation Kit (QBiogene). A control containing only double-distilled H₂O, and no tissue was used. A comparison between amplified products of ODC gene from the research sample preparation to that from the commercial sample is shown in FIG. 8. Finally, human prostate RNA was isolated from epithelial and stromal cells using laser-capture microdissection according to published NIH produces.

ODC Levels Correlate with Prostate Cancer Growth as Measured by PSA Velocity.

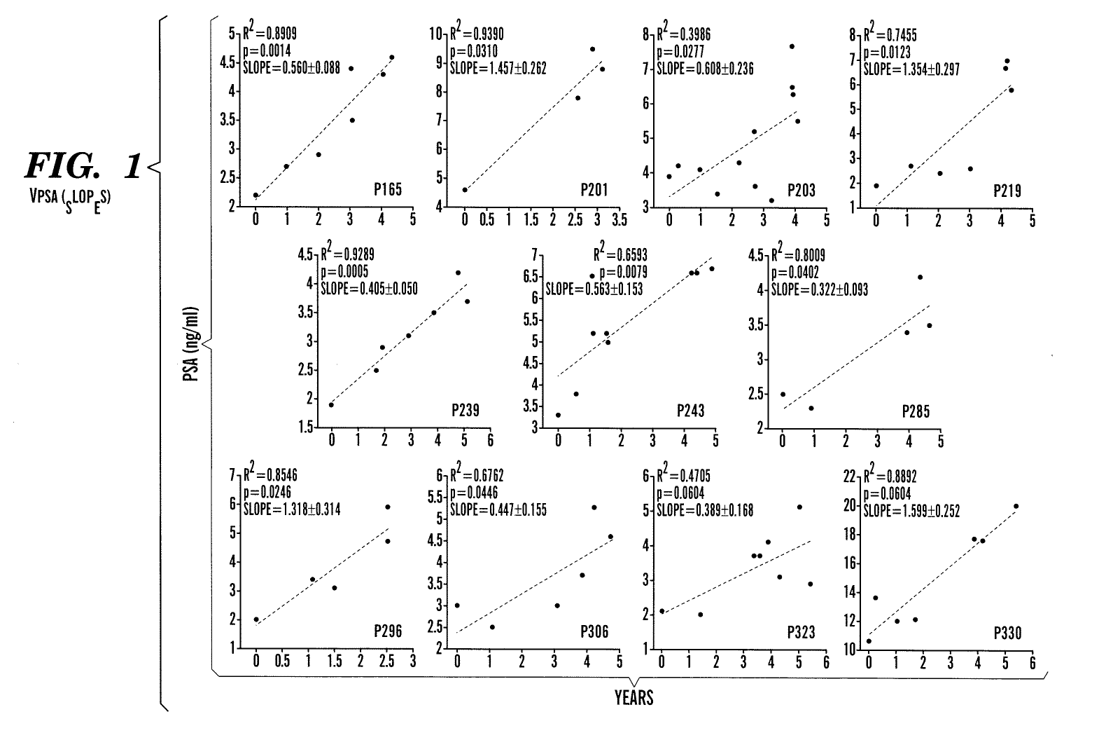

To investigate the relationship between spermine levels, ODC expression and tumor progression, spermine levels and ODC expression levels in tumor tissue was determined for eleven prostate cancer patients treated at MGH with whom multiple blood test results for prostate specific antigen (PSA) prior to prostatectomies were available. PSA velocity, the rate in rise of PSA level over time, prior to diagnosis of prostate cancer, is a powerful indicator of prognosis of the disease, with a high PSA velocity being indicative of poor prognosis, and warranting aggressive therapy. The PSA velocity of each patient was determined by linear regression analysis, and is shown in FIG. 1. These PSA velocities represent the prostate tumor growth rates for each patient.

The amount of spermine in the prostate tissue was analyzed and compared to the tumor growth rate estimated according to the patient serum PSA velocities. No relationship between the spermine levels and tumor growth rate was observed.

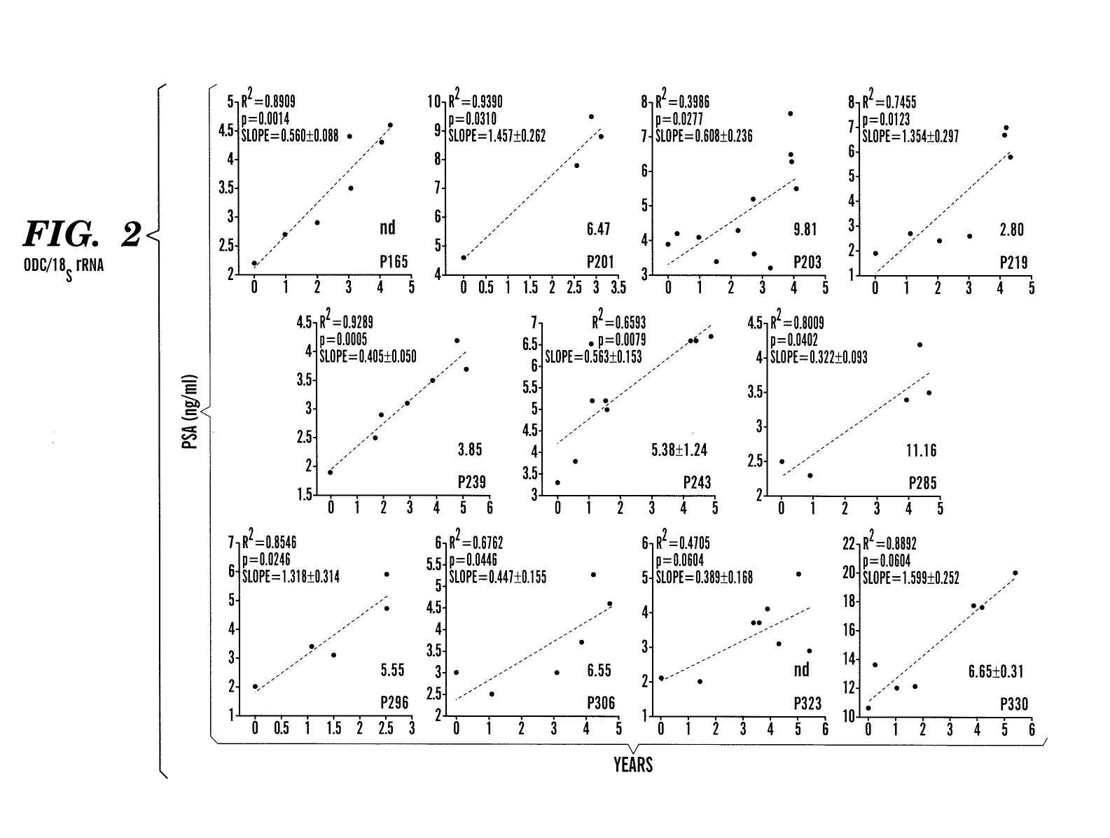

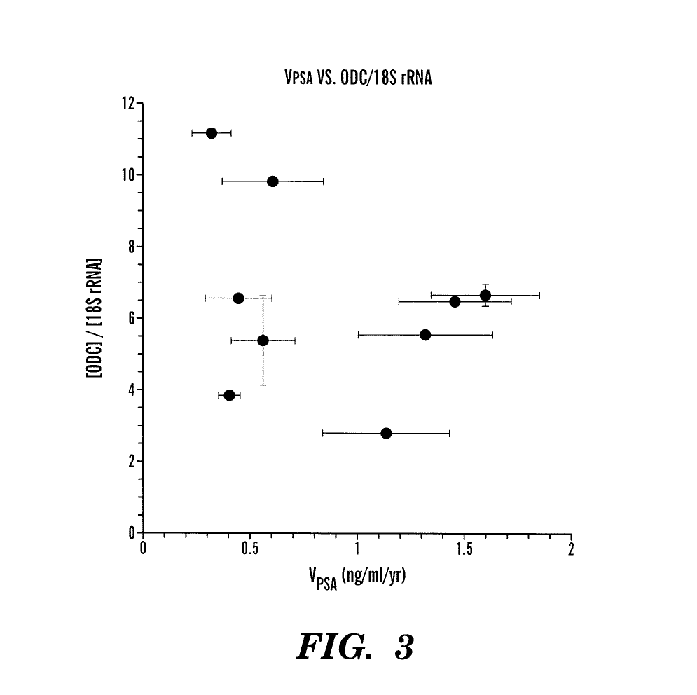

ODC mRNA levels were then investigated. Laser capture microdissection of histologically benign glandular epithelia within the frozen tissue samples obtained from prostatectomy was performed for each patient. This was followed by RT-PCR analysis of the LCM materials for quantitation of ODC mRNA. ODC mRNA was determined as the fold of expression over the house-keeping gene 18S rRNA ([ODC]/[18S rRNA]). FIG. 2 shows the PSA velocity of each patient along with relative ODC mRNA levels. For a few patients, no data on relative ODC mRNA levels was obtainable. The results of data from the nine cases which ODC was measured is shown in FIG. 3, which graphically charts VPSA vs. [ODC]/[18S rRNA].

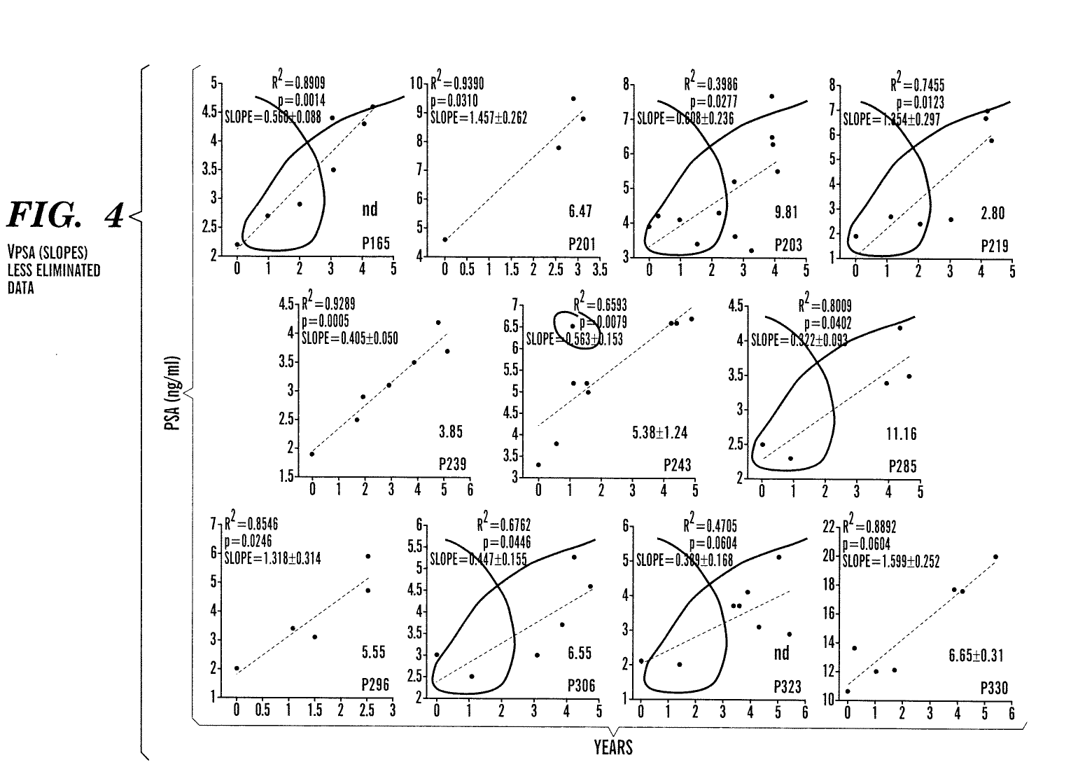

Certain cases were then eliminated from the study based on the following criteria: 1) ODC “nd” cases (P165 and P323); 2) the linearity of less than 85% (P203, P219, and P306). In addition, with the case P243, the clear outlier point (circled) was removed from the final calculation of VPSA. These eliminations resulted in five cases that are used for the final calculation of the relationship between VPSA and [ODC]/[18S rRNA], indicated in FIG. 4.

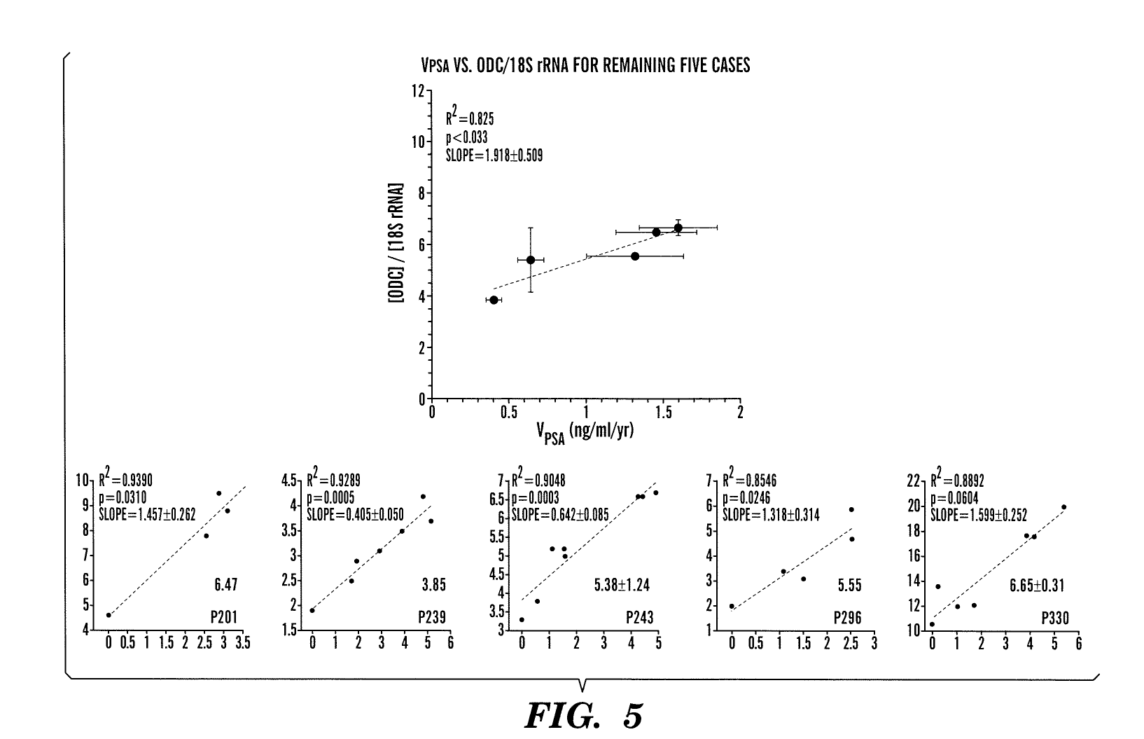

The final observed linear relationship between VPSA and [ODC]/[18S rRNA] for these five cases is indicated in FIG. 5. This plot indicates that by measuring [ODC]/[18S rRNA] at a single clinical point, the prostate tumor growth rate may be predicted and be utilized in clinical decision-making.

The absence of a correlation between spermine levels and tumor progression, but presence of a strong correlation between ODC mRNA and tumor progression, indicates that the dynamic production of spermine in the prostate, rather than the static amount of spermine, is what sustains the growth inhibition. Importantly, this dynamic production of spermine in the prostate is by the histologically-benign prostate epithelia.

The utility of PSA testing in detecting clinically significant prostate tumors has been proven in clinical settings (3-5). However, tumors of clinical significance may be neither lethal to their hosts nor in need of “definitive therapies,” known to be associated with “increased morbidity, particularly incontinence and/or impotence (PRG-p47).” The urgent task is to discover “new and better markers than PSA for the early diagnosis of patients who harbor fast-progressing and virulent forms of prostate cancer (PRG-p31).” These markers are expected to “refine” early detection made with PSA testing by identifying cancers with the potential to kill their hosts if not treated; to “assure” that no virulent cancers are missed; and to improve “prognostic markers that can guide the therapy of patients in an individualized fashion” (PRG-p47). Here, tumor metabolites, measured directly from untreated prostate specimens, are shown to serve as the urgently needed markers for tumor aggressiveness. In particular, the level of ornithine decarboxylase (ODC) is shown to correlate with cancer progression. Currently, without any means of determining prostate tumor growth rate from a positive biopsy result, prostate cancer clinic can hardly differentiate fast grow tumors that need immediate intervention from slow grow tumors that may never become clinical significant. As a result, the latter (representing the majority of the newly diagnosed cases) are over-treated. With this invention, when prostate cancer is detected during a biopsy, if an additional biopsy core can be obtained from which histologically-benign prostate epithelia are isolated (e.g. with LCM), then the level of mRNA of one or more members of the spermine biosynthetic pathway (e.g. ODC mRNA) can be quantitated from these epithelia (e.g. measured by PCR and compared to levels of a house-keeping gene, such as 18sRNA. The relative amount of the enzyme can be used to estimate the rate of the prostate cancer growth, which will contribute to the patient prognosis. With this information, an appropriate treatment can then be determined and followed.

### Evaluation of HRMAS Spectra at Reduced Spinning Rates.

A HRMAS spinning rate of between 2 and 2.5 kHz on the 400 MHz spectrometer was typically utilized. The rationale for choosing this rate range was to push the first order spinning sidebands of tissue water to the outside of metabolite resonance region. This arrangement has proved effective for its simplicity. Histopathological examinations of tissue specimens after spectroscopic studies have shown that such moderate spinning does not severely alter tissue histopathological architectures, and that meaningful pathological evaluations can still be conducted (96, 97). However, spinning-induced reductions in tissue histopathological clarity were also observed. To continue along the path of this rationale, the HRMAS rate for a 600 MHz spectrometer would have to increase to 3 to 3.5 kHz.

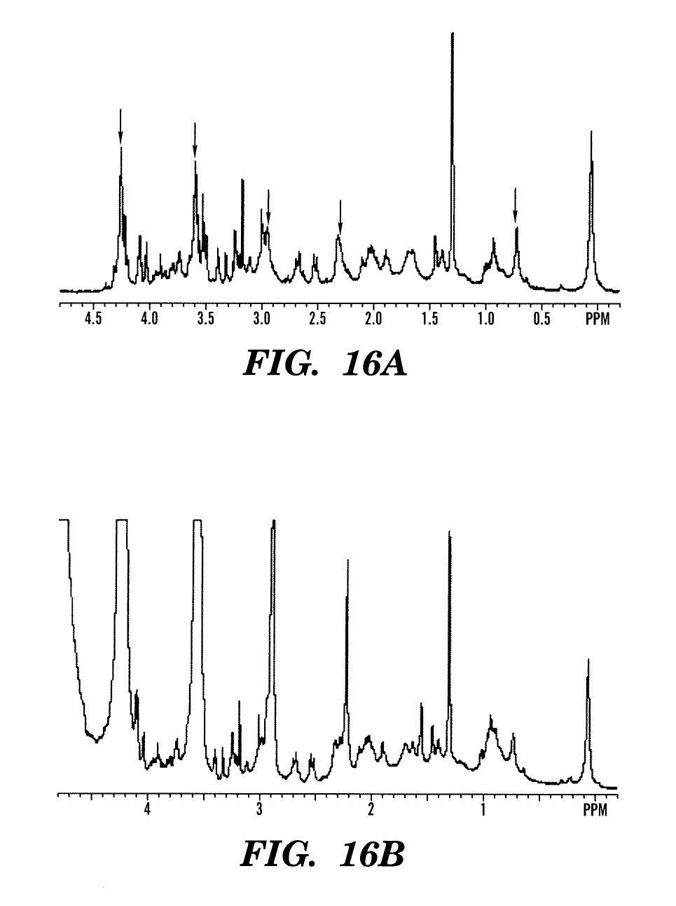

The strength of this project is represented by the sequential analyses of HRMAS spectroscopy and quantitative pathology on the same tissue specimen, as the results obtained from pathological evaluation have extremely important values in establishing cancer metabolic markers. Thus, any compromise to tissue pathology would ultimately weaken a fundamental premise of the analysis. However, by reducing the HRMAS rate, the spinning sidebands from tissue water resonance interfered severely with observation of metabolites (122). Although the application of resonance suppression on tissue water may improve the situation by a certain degree, the resulting spectra were still of limited use. These effects are shown in FIG. 16, which presents prostate tissue spectra obtained with HRMAS rate at 400 Hz both with and without water suppression. To resolve this complication, the feasibility of a solid-state MR pulse sequence called TOSS, total sideband suppression, was tested for its application in prostate tissue. This pulse sequence uses a spinning rate-synchronized five-r pulse scheme to achieve the complete suppression of sidebands (123). Examples of TOSS-water suppressed prostate tissue spectra are shown in FIG. 16.

### Evaluation of the Permanent Standard.

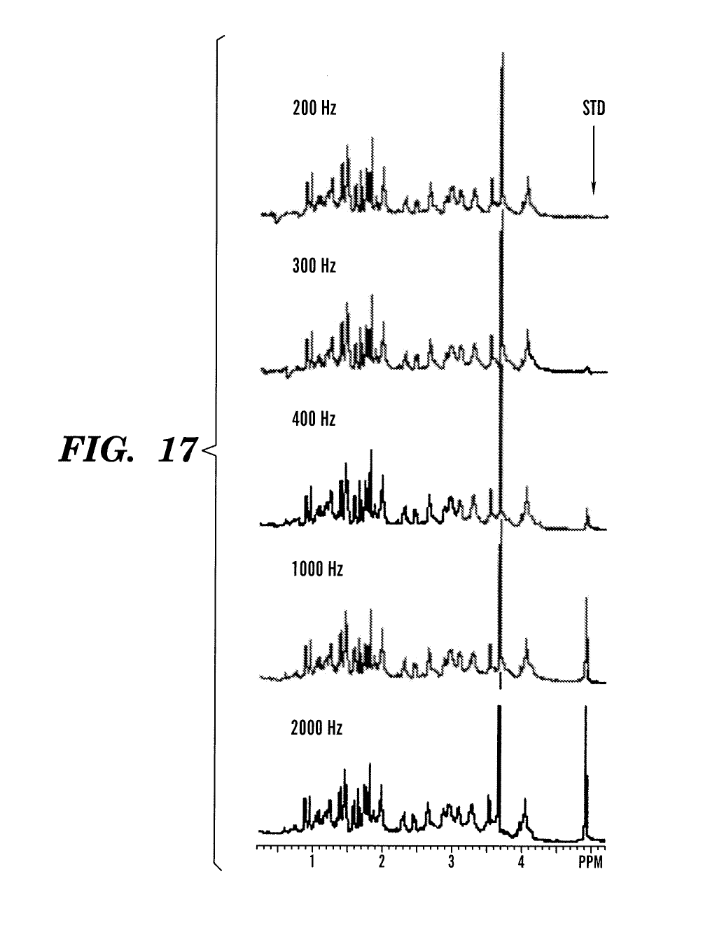

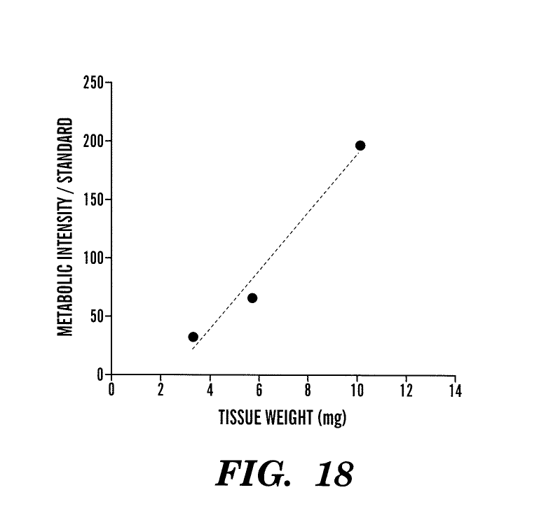

Estimations concerning tissue metabolite concentrations were based on the resonance intensity of tissue water measured from fully recovered spectra without suppression of water resonance. This approach proved effective to provide meaningful results. However, this approach may not work as accurately in a protocol that involves slow spinning rates and TOSS. In addition to the explorations of the relationship between relative metabolite ratios and their clinical values, a permanent silicone rubber standard adhered in the HRMAS rotor was tested for the ability to function as a quantification and resonance reference. The major difference between the standard and biological tissue is that the rubber is indeed in a solid form and has a much great dependency on the spinning rates (see FIG. 17). This figure also indicates that water suppressed TOSS method can produce clean tissue HRMAS spectra for human prostate, and tissue metabolites thus measured do not show dependence on HRMAS rate. As a compromise between the desire to maintain a moderate spinning rate so as to preserve histopathological clarity, and to be able to observe and quantify the standard, multiple prostate samples of varying weight from the same case were tested at a HRMAS rate of 400 Hz (see FIG. 18). Although this plot suggests a linear relationship, the measured slope does not intercept with 0. This may be caused by experimental errors associated with a relatively small standard peak seen at the 400 Hz spinning rate (see FIG. 17).

### Evaluation of Tissue Degradation at Different Temperatures.

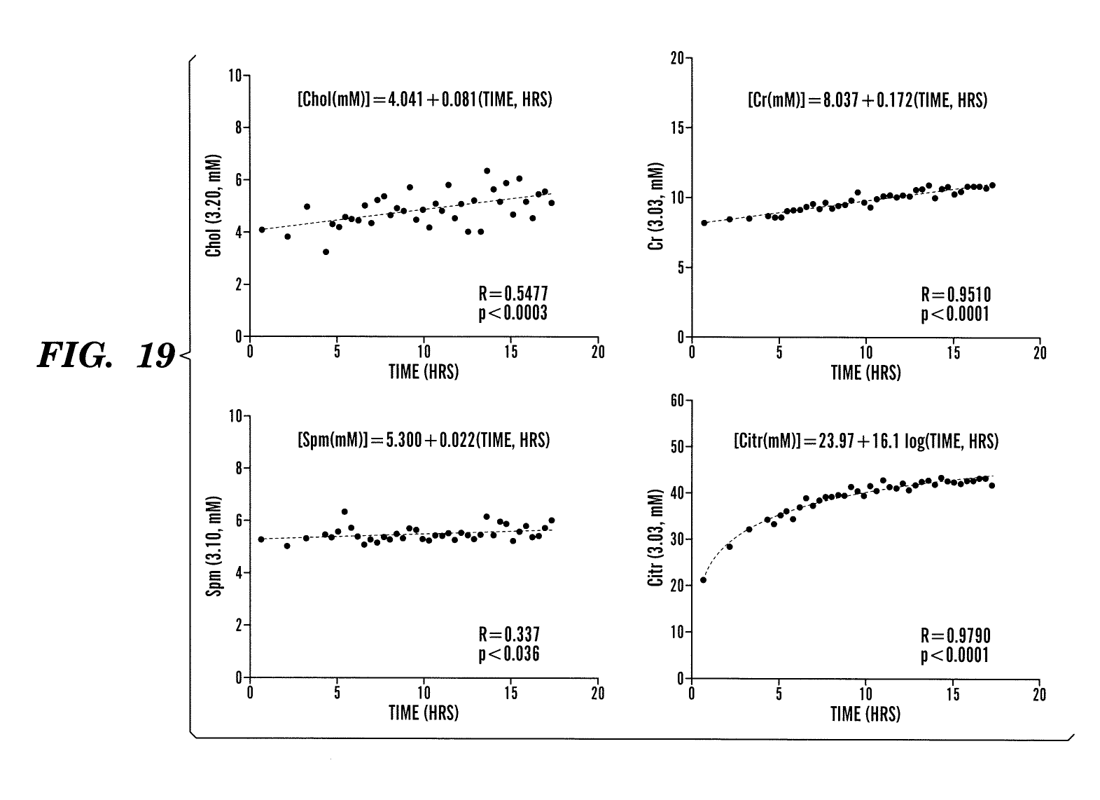

The influence of tissue degradation on the measurement of cellular metabolism must be addressed carefully when the focus of study is metabolite concentration (124). Because metabolic decomposition ensues immediately upon tissue excision, extrapolating the true concentrations of metabolites before tissue excision is of great importance. To estimate the rates of tissue degradation of human prostate, systemic measurements were conducted. Degradation rates of various metabolites found in intact prostate tissue were analyzed by continuously recording prostate tissue HRMAS spectra for 18 hours at 3° C. on a 400 MHz instrument. These experiments were done on both normal and cancerous specimens that had been frozen quickly in liquid N2 in the operating room. Examples of the metabolite degradation curves generated from this analysis are shown in FIG. 19 for choline, spermine, creatine, and citrate.

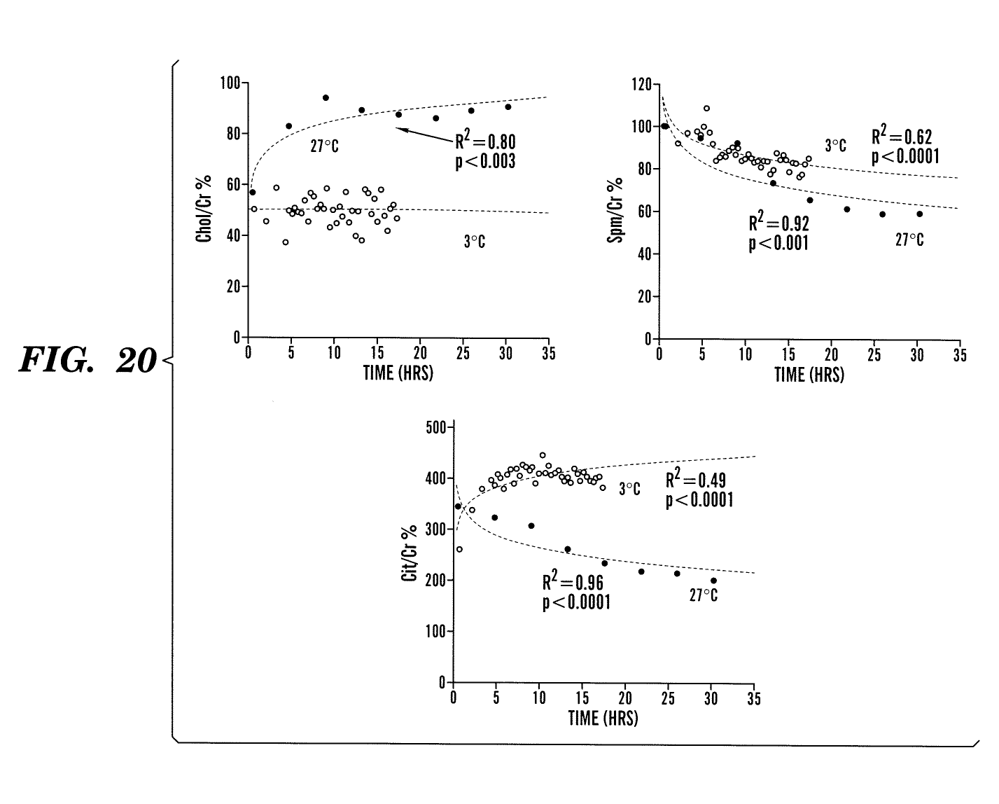

Similar measurements have also been carried out on the MIT 600 MHz spectrometer at 27° C. FIG. 20 compares the relative tissue metabolic changes measured at 27° C. with those obtained at 3° C. (presented in FIG. 19). Metabolic measurements are presented as ratios of the metabolite over creatine, since creatine overall degradation rates are slow and similar at both temperatures (see FIG. 19). The data presented in FIG. 20 indicate that different prostate metabolites have different rates of degradation at different temperatures. If necessary, tissue degradation curves such as these allow the extrapolation of the true metabolite concentrations before tissue excision (or death).

The success of these experiments in developing a biochemistry-based pathology for prostate cancer relies heavily on the establishment of a one-to-one relationship between tissue metabolites that are readily quantifiable by HRMAS 1HMRS and the quantitative pathology features residing in a sample. This places a great demand on the accuracy of quantitative histopathology. If tissue were sectioned less frequently than necessary, the biochemical markers would not be sufficiently sensitive to correlate with quantitative histopathology. However, if the specimens were too frequently sectioned, resources would be wasted (6).

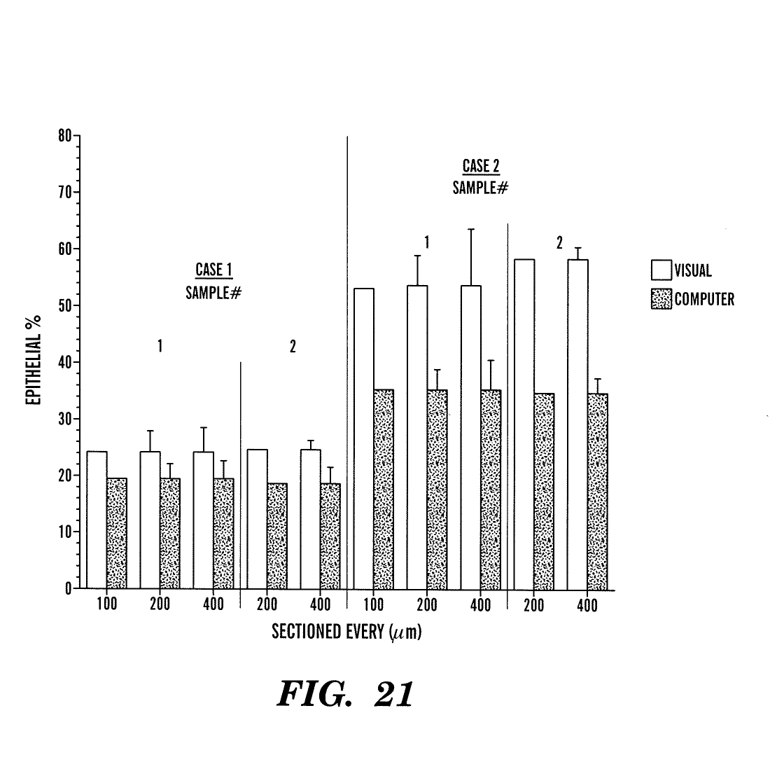

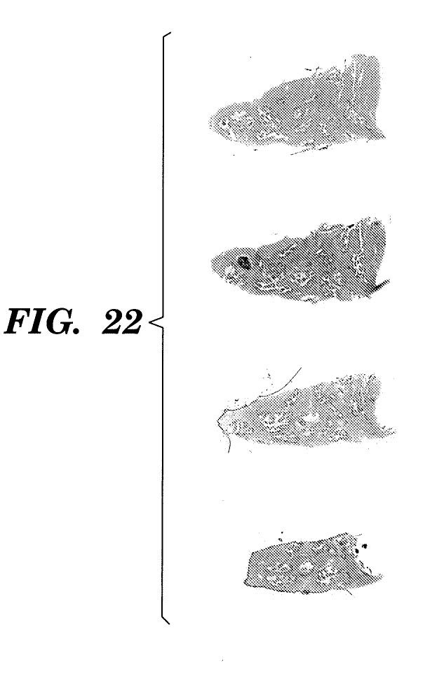

To determine the optimum frequency for a pathology section, a study involving serial sections of two samples from seven cases analyzed by HRMAS 1HMRS was conducted. Serial sections taken at every 100 μm yielded 25 to 50 (an average of 40) slides per sample (20-30 mg). Pathologies were quantified both with visual estimation provided by the pathologist in evaluating the greater than 500 slides in random order, and by computer analysis of digital images. FIG. 21 shows the calculated volume percentage of epithelial cells obtained in this study. The results demonstrate that a section frequency between 200 and 400 μm apart are appropriate for the quantitative histopathology of prostate, since the results measured from slides 200 μm apart did not represent grave deviations from those measured with images acquired every 100 μm. However, when comparing images at 400 μm apart (see examples in FIG. 22), noticeable structural changes were observed. FIG. 21 indicates that previous visual evaluations by the pathologist consistently carried an over-estimation of ˜20%, which must be considered if both visual and computer analyses are involved as parts of a study.

The results herein reported indicate that additional experiments can be performed to further identify correlations between cancer metabolite turnover and prostate cancer progression. These tumor metabolic signatures can be used to develop a new biological paradigm that improves the accuracy of diagnosis and prognosis in prostate cancer clinic and so help direct treatment strategies for an individual patient. For instance. a metabolite database can constructed for prostate cancer according to biological functions of tumor metabolites, which can be continuously updated with patient outcome information. Such future experiments are outlined below.

Autopsy samples from prostate transition, central, and peripheral zones are analyzed. Six prostate samples (two for each zone) from 18 subjects without known prostate disease are collected and evaluated by the project pathologist both before and after these studies to ensure that there are no undetected small focuses of cancer or suspicious cells in these specimens. These 18 subjects are selected evenly from three age groups: <39, 40-59, and >60 years old. The age grouping is based in part on reported incidence for prostate cancer among U.S. males (with less than 1 in 10,000 for men younger than 39 years old; 1 in 57 for men 40-59; and 1 in 6 for men 60-79 years of age (1)) and in part on possible age-related metabolic changes within normal prostate (90).

### Quantifying Metabolic Alterations in Newly Diagnosed Untreated BPH, PIN, and Prostate Adenocarcinomas.

Cancer samples are obtained from the clinic. Importantly, these samples contain sufficient samples from moderately differentiated cases, which represent the most difficult population to evaluate with current pathology, and thus the group most likely to benefit from this approach. Selective emphasis is placed on recruiting well-differentiated tumors (with low Gleason scores) in the sample collection. In addition, at least 10-15 BPHs and PINs are included as part of the proposed 120 cases so as to build up metabolic signatures for the complete spectrum of the transformation of human prostate cancer. In the sample collection, tissue is dissected from prostate transition, central, and peripheral zones if prostatectomy specimens are available for research. In cases where only limited samples are available, such as with BPH, samples are obtained from different zones for different cases, for recordation of zoning information. Such a design is due to the uneven distribution of cancer occurrence (3, 7) and the reported metabolic difference in different zones (76, 125-127). Histopathological features are similar in different zones for both BPH and cancer (24).

### HRMAS 1HMRS Analysis.

For each specimen, approximately one hour of spectrometer time is needed. During this time, two spectra with different lengths of T2 filters (20 and 200 ms) is acquired, and metabolic spin-lattice (T1) and spin-spin (T2) relaxation times are measured. The concentrations for the first 20 most intensive prostate metabolites is calculated by using sample weight in connection with an external reference adhered in the HRMAS rotor and/or by tissue water resonance measured from the same spectrum. Unidentified resonances observed in both normal and tumor tissue HRMAS spectra is investigated and assigned to specific metabolites. To accomplish this task, multinuclear (13C and 31P) and multidimensional MR techniques are employed to elucidate resonance structures.

### Quantitative Pathology.

Quantitative pathology is conducted on fresh-frozen or fixed prostate tissues serially sectioned at 200 μm intervals after the spectroscopy analysis. The following epithelial features are quantified as the volume percentage of the entire specimen examined: atrophy, normal, proliferative, pre-neoplastic (prostatic intraepithelial neoplasm), and malignancy. In addition, the amount of epithelial cells is presented in terms of cellular density (cells/mm³), and inflammation as well as stromal proliferation is graded.

### Metabolic Markers for the Prostate.

Quantitative pathology reports are used to correlate tumor histopathological features with metabolite concentration obtained from the HRMAS 1HMRS analysis. Alterations in metabolite concentration arising from the histopathological features of normal, BPH, PIN, and cancer are statistically analyzed by multivariate, multiple regression. Metabolites that can significantly differentiate between normal and BPH from any two grades of tumors (including PIN) are evaluated for their sensitivity in distinguishing tumor grade. Values of the mean of absolute metabolite concentrations that satisfy this criterion (i.e., by differentiating tumors from normal and BPH and distinguishing between two types of tumors) are considered as biochemical markers for the corresponding two grades.

### Statistical Analyses for Normal Prostate.

Normal tissue from three prostate zones of human subjects in three age groups are analyzed to establish metabolic databases for normal human prostate. The mean values of the absolute metabolite concentrations in each tissue category are calculated. A two-way ANOVA is used to analyze the effects of age group and prostate zone upon the mean valve of each metabolite concentration. Additionally, for each prostate zone, a one-way ANOVA is performed to compare the mean values of the age-groups for each metabolite. If there is a statistically significant separation of the three tissue types from different zones in the three-way ANOVA, and a statistically significant difference among age groups for each metabolite measured (by zone), no more normal subjects are studied in the future. Otherwise, power calculations are performed to determine the scale of future studies, or to conclude that age-related metabolic variations may not be observable by using the current methodology.

### Statistical Analyses for Tumor and BPH Specimens.

The statistical analysis of data tests the hypothesis that the mean metabolite concentrations of tissue measured by HRMAS 1HMRS differ according to types of tissue and tumor histopathology. The relationship between the extent of each tumor histopathological feature and the measured absolute metabolite concentrations are analyzed by using multivariate, multiple regression. These analyses include the above-mentioned quantities of the eight histopathological features and the first 20 most intensive metabolites. These multiple regression analyses are conducted by using the eight histopathological features as independent variables with 120 points (the number of tumor cases including the existing samples and future collections) for each metabolic concentration. Assuming that R2 is at least 0.20, each analysis, with a significance level set at 0.05/20=0.0025 where 20 represents the total number of metabolites analyzed, can provide 80% or better power. To avoid the problems of multiple comparisons that analyze from independently analyzing 20 metabolite concentrations, multivariate analysis is used to analyze them simultaneously, thereby avoiding the chance of coincidental Type I errors.

The sensitivity and specificity of prostate metabolic markers in predicting tumor pathologies, tumor grades, and pathological stages is also assessed with pseudo-biopsy samples.

Evaluation of Prostate Metabolic Markers for Evaluations of Clinical Relevance of Prostate Metabolic Markers Identified from Studies Conducted Above.

This research consists two parts: assessing the ability of metabolic markers in predicting prostate pathologies and tumor grades, and their sensitivity and specificity in predicting pathological stage with pseudo-biopsy samples. In addition to tumor grade, the pathologic stage is another, even more, important factor that directs prostate cancer therapy. Currently, pathologic staging can only be achieved through prominent section of at least 20 samples from the whole prostate after prostatectomy. Since metabolite markers reflecting tumor biological activities may be more sensitive than tumor morphology and may present prior to morphological changes, metabolite markers, observed with pseudo-biopsy samples collected post-surgically from prostatectomy specimens according to the standard eight-site sampling procedure, are expected to be sensitive enough to indicate cancer pathologic stage. The success of this results in the ability of predicting pathologic stage from biopsy and reduction in the number of unnecessary prostatectomies.

90 cases including all types of prostate samples analyzed in above (from normal to the most malignant adenocarcinoma) are used to determine sensitivity and specificity for each marker. These 90 cases, forming a test set, are analyzed according to the same protocol as discussed above. Histopathological information on the specimens is not revealed until the conclusion of each spectroscopic diagnosis. Upon completion of HRMAS 1HMRS and quantitative pathology for the 90 samples, biochemical markers for each grade of tumor are reevaluated to accommodate the newly measured data. The sensitivity and specificity of tumor markers is analyzed for each grade of adenocarcinoma according to tissue pathologies measured after HRMAS spectroscopy, the clinical data, and the methods outlined below. If neither sensitivity and/or specificity of the markers for one grade of tumor is greater than 90%, a power calculation is performed to determine the number of additional tumor samples that should be added to the databases in future investigations.

### Sensitivity and Specificity Analyses.

Sensitivity and Specificity is determined by the following formulas (128) with terms defined in Table I:

TABLE I
Definition of terms used in the evaluation of sensitivity and
specificity.

Tumor Pathology Status

| | | True | False |
|---|---|---|---|
| Spectroscopic | Positive | TruePositive | FalsePositive |
| Results | Negative | TrueNegative | FalseNegative |

$Sensitivity = \frac{TruePositive}{TruePositive + FalseNegative } * 100 \% and$
$Specificity = \frac{TrueNegative}{TrueNegative + FalsePositive} * 100 \%$

Pseudo-biopsy for pathological staging. Among these 90 cases, 60 are selected from which both pseudo-biopsy samples with a biopsy gun from prostates after prostatectomy are collected according to the exact procedure used in surgery, and at least additional 20 samples are collected following the exact sampling protocol currently directing the practice of the permanent histopathological sectioning for prostate cancer staging. The majority of the 60 cases come from the population of moderately differentiated tumors because of the critical value of their pathological stages in the determination of treatment strategies.

Prostate metabolites are measured with samples from both eight-site pseudo-biopsy and 20-site permanent histopathological sections from the same case. Quantitative histopathology on these samples after their spectroscopy studies are examined against the clinical reports on pathological diagnosis. Agreement between the means of prostate metabolites measured from samples of pseudo-biopsies and permanent sections are evaluated for individual prostate zones. Complete agreement between the two means is modeled by an ideal linear function with a slope of 1.0 and an intercept of 0.0. Metabolites that result in slopes within 10% of the ideal of 1.0, e.g. 0.9 to 1.1, are considered as having the ability of representing permanent sections with biopsies. The test of the relationship between the two means for departure from this ideal function is designed to detect those metabolites that produce a departure>10%.

The statistical significance for each metabolite in the differentiation of cancer from BPH and distinguishing among tumor groups (i.e. diagnostic evaluation), as well as in predicting tumor clinical stage, is determined by a one-way analysis of variance.

Metabolic pathways and functions for tumor markers are measured by using biomolecular technologies including RT-PCR on LCM prostate materials to quantify the influence of prostate metabolites, such as spermine and citrate, on tumor growth and progression as evaluated in clinic.

The relationship among the apparent spermine concentration in epithelial cells (determined by HRMAS-measured spermine concentration versus total amount of epithelial cells from quantitative pathology), activities and/or expression levels of enzymes involved in spermine synthetic pathway, including ODC, SRM, SMS, SMO, SSAT, AND PAO, (observed with LCM/RT-PCR for normal epithelial and cancer cells and other cellular components), and clinical progression rate (measured as the PSA velocity (4)) are further analyzed. This population is the starting point because there is a higher frequency of multi-PSA testing results recorded in patients' medical histories for this group compared with any other group, from which PSA velocity is calculated, and because of the diverse disease outcome associated with this group. It is estimated that about 70-80 of planned collections of 120 cases belong to this population, and about ⅓ of these 70-80 cases (i.e. 20-25 cases) have multi-PSA testing (n>3 or 4). The results from measurements of these cases allow the design of specific biostatistical models to evaluate the relationship between epithelial spermine concentrations and/or ODC activities with progression rates, as discussed below.

A metabolite database is constructed for prostate cancer according to biological functions of tumor metabolites, which is continuously updated with patient outcome information. In addition, a spectroscopic protocol is designed that is aimed at evaluating tumor biochemical behavior and detecting life-threatening moderately differentiated adenocarcinomas in clinic. Metabolite and patient outcome data is tabulated in a database. Patient outcome data is modified semi-annually. The following patient information should be recorded: age, symptoms (DRE, PSA, etc) site (zone) of tumor (determined from operative notes), multi-PSA testing results if available prior to surgery, type of operation, initial diagnosis (Gleason grade and clinical stage), final diagnosis (Gleason grade and pathologic stage), adjuvant therapy (irradiation, chemotherapy, and subsequent operations), date of operation, date of death or last follow-up, and cause of death (see Appendix for a data form). Metabolite markers identified by HRMAS 1HMRS are tested for correlation with patient survival by Kaplan-Meier Survival Analysis and proportional hazards regression models (134-136).

### Selection of Patients and Sampling Mechanism

Specimens used for this study are examined, selected and coded by the project pathologist and type blinded. These coded samples are stored at −80° C. in the lab until they are ready for spectroscopic analysis. Upon completion of HRMAS 1HMRS measurement, each specimen is transferred to the project pathologist for quantitative histopathological evaluation. The pathologist will produce pathological reports on the specimen. The pathology reports and codes identifying each sample will be maintained by the project pathologist. Upon completion of the specified time interval for the spectroscopic studies, the analyzed spectroscopic results, sample identification code and pathology report will be submitted simultaneously to the project statistician for analysis.

### Normal Prostate.

Autopsy specimens less than 18 hours post-mortem obtained from subjects, without known prostate-related diseases are evaluated by pathologist qualitatively before and quantitatively after HRMAS 1HMAS as normal controls. Samples are collected from three age groups (<39, 40-59, and >60), with six subjects in each age group. For each subject, two tissue specimens (20-50 mg) are dissected from three prostate zones: transition, central, and peripheral. Adenocarcinoma Metabolism and LCM/RT-PCR.

This group includes 60+ cases of specimens of human prostate adenocarcinomas already collected and additional 120 samples to be collected. These 120 cases are selected to include 10-15 cases of each type of BPH, PIN, and well-differentiated tumors (Gleason score<5). The project pathologist provides these coded samples to the PI without revealing information regarding their histopathological status. The pathologist randomly selects 30˜60 mL specimens for the HRMAS 1HMRS analysis, while preserving the adjacent block in −80° C. for future usage if necessary. HRMAS 1HMRS measurements of these samples is performed blindly. Finally, after HRMAS 1HMRS, the pathologist performs quantitative histopathological measurements on the specimens and produces reports. It is expected that within these 120 cases, about 20-25 of them are moderately differentiated tumors that also have patient medical histories of multi-PSA testing. These cases are recruited for the LCM/RT-PCR analysis of their ODC activities in different prostate histology components.

### Evaluation of Biochemical Markers.

Ninety cases consisting of normal, BPH, PIN tissues, and adenocarcinomas of different Gleason scores are selected by project pathologist. These specimens are used for the assessment of the diagnostic sensitivity and specificity of tumor biochemical markers. Thirty ˜60 mL samples are provided by the pathologist and coded without further identification. HRMAS 1HMRS measurements are obtained and quantitative histopathology performed. Of these 90 cases, samples from 60 cases are collected both as pseudo-biopsy samples with a biopsy gun following the eight-site procedure from prostates after prostatectomy and as permanent histopathological sections with at least 20 samples according to the established sampling protocol of prostate pathology. These pseudo-biopsy samples are used to test the sensitivity and specificity of metabolic markers in predicting pathological stage of prostate cancer that are currently achieved with permanent sectioning after prostatectomy.

### HRMAS Proton MR Spectroscopy

Specimens are weighed before the HRMAS 1HMRS and transferred to a HRMAS sample rotor containing a permanently adhered external standard (silicone rubber) that functions as a reference both for resonance identification and quantification. The HRMAS 1HMRS measurement is carried out at 3° C. to minimize tissue degradation during the measurement. Spectra is acquired on a 600 MHz Bruker spectrometer, with a rotor-synchronized Carr-Purcell-Meibom-Gill (CPMG) pulse sequence, [90-(τ-180-τ)ₙ-acq] and a HRMAS rate of 3.0 kHz, or with a rotor-synchronized total sideband suppression (TOSS) five-π pulse sequence (123) at HRMAS rates between 400-1000 Hz, if protection of tissue pathological structures from damages caused by high speed spinning is necessary. For CPMG, the inter-pulse delay (τ=2π/ωᵣ) is synchronized to the rotor rotation. By varying the value of n, we will use a T2 filter of 20 ms. For TOSS, each rotor cycle reciprocally represents T2 filter time. Additional filter time can be achieved with multiple TOSS cycles. The 256 transients are acquired with a spectral width of 12 kHz and 16 k data points, and a repetition rate of 5 sec. After HRMAS 1HMRS spectroscopy, specimens are transferred to the project pathologist for quantitative histopathology.

To elucidate resonance structures, homo- and heteronuclear correlation spectroscopy of intact specimens are acquired under HRMAS conditions. For these tests, both with and without CPMG, a spectral width of 10 ppm and a repetition rate of 1 second is used. The time of one measurement will be kept within 3 to 4 hours at 3° C. to minimize possible metabolic degradation (92). Carbon-13 and 31P heteronuclear correlation spectroscopy can provide more precise isotropic chemical shift values. “Constant-time” J-spectroscopy is applied in the proton homonuclear experiments: the 11-modulation caused by the variation of the position of π-pulse will only affect the chemical shift, but not the homonuclear coupling, and achieve the separation of interactions (137). The heteronuclear correlation spectroscopy (π/2-τ1-π/2-τ2), where the second π/2 pulse is applied to both proton and carbon, are used in the study of proton-carbon coherence transfer pathways (138). These studies not only provide better separated spectra due to the much wider carbon-13 chemical shift range (200 ppm for 13C vs. 10 ppm for 1H), but also map out chemical structures (99, 139-141).

### Spectral Analysis.

Spectra are analyzed by both Bruker software, equipped with the spectrometer, and the Nuts program produced by Acorn NMR for Windows systems. Before Fourier transformation and phasing, all free induction decays are subjected to 1 Hz apodization. The 20 most intense resonances in the spectra are measured by integrating appropriate resonances relative to the external and internal standards of tissue water. These resonances are identified and assigned to corresponding metabolites; their intensities are used to quantify the metabolites.

### Histopathological and Image Analyses

Upon completion of the HRMAS analysis, all tumor histopathological analyses are performed on either formalin-fixed and paraffin-embedded, or frozen-embedded specimens processed according to the routine pathology procedures. For such examinations, specimens are serially sectioned (200 μm apart) on a microtome or a cryostat at 5 micron thickness and stained with Hematoxylin and Eosin. The entire specimen is prepared for evaluation that will result in approximately 10 to 15 pathology slides for each specimen. These sections are then examined by the project pathologist to correlate the cellular composition of the specimen with the HRMAS data.

Computer-aided quantitative analysis with software NIH-Imaging is conducted on digital microscopic images. Each tumor specimen is quantitatively examined according to the following epithelial features: atrophy, normal, proliferative, pre-neoplastic (prostatic intraepithelial neoplasm, PIN), and malignancy. The proportion of these features are quantified as a percentage of total specimen volume, and the amount of epithelial cells are presented as cellular density (cells/mm3). In addition, inflammation and stromal proliferation are also systematically graded on a scale of 1 to 10. Under certain tissue/cellular conditions, such as in cases of many infiltrating tumors where clear borders between different structures may be difficult to establish. If such a condition raises to a concern, the procedure can be modified to develop and implement an object and/or color-oriented quantitation process with the assistance of commercial image analyzing software, such as Image-Pro® from Media Cybernetic® (Silver Spring, Md.).

RT-PCR Amplification of ODC mRNA

Laser Capture Microdissection. Prostate biopsy samples are frozen-embedded in O.C.T. (TissueTek) and sectioned 7 μm onto uncharged glass specimen slides. Immediately prior to laser-capturing, the slides are stained with Hematoxylin and Eosin, according to the NIH LCM Protocol. Stained slides are then placed under a Pixcell II LCM microscope (arcturus) and, using 1000 pulses for each cell type with a beam width of 7.5 μm, cellular materials from the same sample are isolated and transferred to separate High Sensitivity LCM Transfer film caps (arcturus).

### RNA Extraction and Isolation.

The cap is fitted onto the rim of a 0.5 mL Eppendorf tube for RNA extraction and isolation using the RNA Micro-Isolation Kit from Stratagene, a guanidinium isothiocyanate (GITC) and phenol-chloroform extraction-based method. The tube containing 100 μL RLT buffer (Qiagen) is inverted and incubated in an oven for 15 minutes at 42° C. for 15 minutes to digest materials on the film. The buffer volume is increased to 200 μL and transferred to a 1.5 mL Eppendorf and a phenol-chloroform extraction is performed (20 μL 2M sodium acetate, 220 μL water-saturated phenol, and 60 μL chloroform-isoamyl alcohol, vortexed and placed on ice for 15 minutes, centrifuged at 4° C. for 30 minutes and the top layer transferred to a new 1.5 mL Eppendorf). The nucleic acid is then precipitated (2 μL glycogen, 200 μL cold isopropanol, stored at −80° C. over night, centrifuged at 4° C. for 30 minutes, and the supernatant removed). The glycogen pellet is washed (2×200 μL cold DEPC-treated 70% Ethanol and vacuum-dried), DNAse treated with a GenHunter DNAse Kit (16 μL DEPC-H₂O, 1 μL RNAse inhibitor (RNAsin, Promega), 2 μL 10×Rxn buffer, 1 μL DNAse I), and incubated at 37° C. for 30 minutes. The RNA is then re-extracted, precipitated and washed as above. The RNA pellet is finally resuspended in DEPC-treated RNAse-free H₂O (8 μL) for RT-PCR.

### RT-PCR Analysis.

Reactions are carried out with Superscript one-step RT-PCR Kit (25 μL 2×Rxn mixture, 1 μL sense primer, 1 μL antisense primer, 4 μL laser-captured RNA target, 18 μL DEPC-treated H₂O, 1 μL RT/platinum Taq polymerase). In addition to ODC primers (121), primers for the so-called “housekeeping gene” GAPDH are used as a positive control to test the success of the LCM and extraction method. After a RT-PCR method is carried out according to the manufacturer's protocol, the samples are analyzed by gel electrophoresis on an Agarose gel (2% Agarose in TAE buffer, 100 V for 1 hour). Quantitation of RT-PCR results is achieved with Kodak EDAS 290 Digital Imaging System and its quantitation software.

By using metabolic markers measured directly from intact human prostate specimens, harmless (indolent) prostate tumors are distinguished from potentially lethal ones, and those tumors likely to progress to a life-threatening stage within a patient's lifetime are identified. This task is accomplished using high resolution magic angle spinning (HRMAS) proton magnetic resonance spectroscopy (1HMRS), an objective and non-destructive modality that sensitively measures subtle, cellular biochemical changes accompanying tumor development and progression in untreated prostate specimens, coupled with quantitative histopathology evaluation and the molecular biology analysis of cells, isolated by laser capture microdissection (LCM). By relating spectroscopic results with quantitative histopathology and clinical status, metabolic markers are established for normal prostate, benign prostate hyperplasia (BPH), prostatic intraepithelial neoplasm (PIN), and prostate adenocarcinomas. These markers are used to predict tumor pathological grade and stage, and thus a more accurate diagnoses and prognoses.

By correlating spectroscopic results with LCM bio-molecular analysis of prostate pathologies, the nature of prostate metabolites are defined, and their functions in tumor development and progression discovered. An understanding of the biomedical basics of these metabolic markers allows a more specific patient prognostication than was previously possible, and thus assists clinicians in determining the most effective, appropriate course for the management of cancer, and in particular, prostate cancer in individual patients.

### REFERENCES

1. Greenlee, R. T., Hill-Harmon, M. B., Murray, T., and Thun, M. Cancer Statistics, 2001. CA Cancer J Clin, 51: 15-36, 2001.
2. von Eschenbach, A. The challenge of prostate cancer. CA Cancer J Clin, 49: 262-263, 1999.
3. Carroll, P., Lee, K., Fuks, Z., and Kantoff, P. Cancer of the prostate. In: V. DeVita, Jr, S. Hellman, and S. Rosenberg (eds.), Cancer Principles & Practice of Oncology, pp. 1418-1479. Philadelphia: Lippincott-Raven, 2001.
4. Brawer, M. K. Prostate-specific antigen: current status. CA Cancer J Clin, 49: 264-281., 1999.
5. Brawer, M. K. Prostate-specific antigen. Semin Surg Oncol, 18: 3-9., 2000.
6. Hamilton, P. W., Bartels, P. H., Montironi, R., Anderson, N. H., Thompson, D., Diamond, J., Trewin, S., and Bharucha, H. Automated histometry in quantitative prostate pathology. Anal Quant Cytol Histol, 20: 443-460., 1998.
7. Oesterling, J., Fuks, Z., Lee, C., and Scher, H. Cancer of the prostate. In: V. DeVita, Jr, S. Hellman, and S. Rosenberg (eds.), Cancer Principles & Practice of Oncology, pp. 1322-1386. Philadelphia: Lippincott-Raven, 1997.
8. Bubendorf, L. High-throughput microarray technologies: from genomics to clinics. Eur Urol, 40: 231-238., 2001.
9. Latil, A. and Lidereau, R. Genetic Aspects of Prostate Cancer. Virchows Arch, 432: 389-406, 1998.
10. Mohan, R. R., Challa, A., Gupta, S., Botswick, D. G., Ahmad, N., Agarwal, R., Marengo, S. R., Amini, S. B., Paras, F., MacLennan, G. T., Resnick, M. I., and Mukhtar, H. Overexpression of Ornithine Decarboxylase in Prostate Cancer and Prostatic Fluid in Humans. Clinical Cancer Research, 5: 143-147, 1999.
11. Mi, Z., Iramer, D., Miller, J., Bergeron, R., Bemacki, R., and Porter, C. Human prostate carcinoma cell lines display altered regulation of polyamine transport in response to polyamine analogs and inhibitors. Prostates, 34: 51-60, 1998.
12. Paweletz, C. P., Charboneau, L., Bichsel, V. E., Simone, N. L., Chen, T., Gillespie, J. W., Emmert-Buck, M. R., Roth, M. J., Petricoin, I. E., and Liotta, L. A. Reverse phase protein microarrays which capture disease progression show activation of pro-survival pathways at the cancer invasion front. Oncogene, 20: 1981-1989., 2001.
13. Still, K., Robson, C. N., Autzen, P., Robinson, M. C., and Hamdy, F. C. Localization and quantification of mRNA for matrix metalloproteinase-2 (MMP-2) and tissue inhibitor of matrix metalloproteinase-2 (TIMP-2) in human benign and malignant prostatic tissue. Prostate, 42: 18-25., 2000.
14. Mountford, C., Doran, S., Lean, C., and Russell, P. Cancer pathology in the year 2000. Biophys. Chem., 68: 127-135, 1997.
15. Stamey, T. A., Freiha, F. S., McNeal, J. E., Redwine, E. A., Whittemore, A. S., and Scmid, H. P. Localized prostate cancer. Relationship of tumor volume to clinical significance for treatment of prostate cancer. Cancer, 71: 933-938., 1993.
16. McNeal, J. E. Prostatic microcarcinomas in relation to cancer origin and the evolution to clinical cancer. Cancer, 71: 984-991., 1993.
17. Rossi, M. C. and Zetter, B. R. Selective stimulation of prostatic carcinoma cell proliferation by transferrin. Proc Natl Acad Sci USA, 89: 6197-6201., 1992.
18. Gleason, D. Classification of prostatic carcinomas. Cancer Chemother. Rep., 50: 125, 1966.
19. Broders, A. Epithelium of the genito-urinary organs. Ann. Surg., 75: 574, 1922.
20. Mostofi, F., Davis, C., and Sesterhenn, I. Pathology of carcinoma of the prostate. Cancer, 70: 235, 1992.
21. Gaeta, J., Asiwartham, J., and Miller, G. Histologic grading of primary prostate cancer: a new approach to an old problem. J. Urol., 123: 689, 1980.
22. Bocking, A., Kiehn, J., and Heinzel-Wach, M. Combined histologic grading of prostatic carcinoma. Cancer, 50: 288, 1982.
23. Brawn, P., Ayala, A., and von Eschenbach, A. Histological grading of prostate adenocarcinoma: the development of a new system and comparison with other methods. Cancer, 49: 525, 1982.
24. Cotran, R., Kumar, V., and Robbins, S. Pathologic basis of disease, 5th edition. Philadelphia: W.B. Saunders, 1994.
25. Gleason, D. Histologic grading of prostate cancer: a perspective. Hum. Pathol., 23: 273, 1992.
26. Partin, A., Kattan, M., Subong, E., Walsh, P., Wojno, K., Oesterling, J., Scardino, P., and Pearson, J. Combination of prostate-specific antigen, clinical stage, and Gleason score to predict pathological stage of localized prostate cancer. A multi-institutional update [see comments]. JAMA, 277:1445-1451, 1997.
27. Signoretti, S., Waltregny, D., Dilks, J., Isaac, B., Lin, D., Garraway, L., Yang, A., Montironi, R., McKeon, F., and Loda, M. p63 is a prostate basal cell marker and is required for prostate development. Am J Pathol, 157: 1769-1775., 2000.
28. Cohen, R. J., Holland, J. W., Redmond, S. L., McNeal, J. E., and Dawkins, H. J. Identification of the glycosaminoglycan keratan sulfate in the prostatic secretory cell. Prostate, 44: 204-209., 2000.
29. Dawkins, H. J., Sellner, L. N., Turbett, G. R., Thompson, C. A., Redmond, S. L., McNeal, J. E., and Cohen, R. J. Distinction between intraductal carcinoma of the prostate (IDC-P), high-grade dysplasia (PIN), and invasive prostatic adenocarcinoma, using molecular markers of cancer progression. Prostate, 44: 265-270., 2000.
30. Recker, F., Kwiatkowski, M. K., Piironen, T., Pettersson, K., Huber, A., Lummen, G., and Tscholl, R. Human glandular kallikrein as a tool to improve discrimination of poorly differentiated and non-organ-confined prostate cancer compared with prostate-specific antigen. Urology, 55: 481-485., 2000.
31. Liu, A. Y., True, L. D., LaTray, L., Nelson, P. S., Ellis, W. J., Vessella, R. L., Lange, P. H., Hood, L., and van den Engh, G. Cell-cell interaction in prostate gene regulation and cytodifferentiation. Proc Natl Acad Sci USA, 94: 10705-10710., 1997.
32. Liu, A. Y., LaTray, L., and van Den Engh, G. Changes in cell surface molecules associated with in vitro culture of prostatic stromal cells. Prostate, 44: 303-312., 2000.
33. Elsasser-Beile, U., Przytulski, B., Gierschner, D., Grussenmeyer, T., Katzenwadel, A., Leiber, C., Deckart, A., and Wetterauer, U. Comparison of the activation status of tumor infiltrating and peripheral lymphocytes of patients with adenocarcinomas and benign hyperplasia of the prostate. Prostate, 45: 1-7., 2000.
34. Fry, P. M., Hudson, D. L., O'Hare, M. J., and Masters, J. R. Comparison of marker protein expression in benign prostatic hyperplasia in vivo and in vitro. BJU Int, 85: 504-513., 2000.
35. Liu, A. Y. Differential expression of cell surface molecules in prostate cancer cells. Cancer Res, 60: 3429-3434., 2000.
36. Ghossein, R. A. and Bhattacharya, S. Molecular detection and characterisation of circulating tumour cells and micrometastases in solid tumours. Eur J Cancer, 36: 1681-1694., 2000.
37. Belair, C. D., Yeager, T. R., Lopez, P. M., and Reznikoff, C. A. Telomerase activity: a biomarker of cell proliferation, not malignant transformation. Proc Natl Acad Sci USA, 94: 13677-13682., 1997.
38. Smith, R., Litwin, M., Lu, Y., and Zetter, B. Identification of an endogenous inhibitor of prostate carcinoma cell growth. Nature Med, 1: 1040-1045, 1995.
39. Higuchi, C. and Wang, W. Comodulation of cellular polyamines and proliferation: biomarker application to colorectal mucosa. J. Cell. Biochem., 57: 256-261, 1995.
40. Fernandez, C., Sharrard, R., Talbot, M., Reed, B., and Monks, N. Evaluation of the significance of polyamines and their oxidases in the aetiology of human cervical carcinoma. Br. J. Cancer, 72: 1194-1199, 1995.
41. Notarnicola, M., Linsalata, M., Caruso, M., Cavallini, A., and Di Leo, A. Low density lipoprotein receptors and polyamine levels in human colorectal adenocarcinoma. J. Gastroenterol., 30: 705-709, 1995.
42. Nishioka, K., Melgarejo, A., Lyon, R., and Mitchell, M. Polyamines as biomarkers of cervical intraepithelial neoplasia. J. Cell. Biochem. Suppl., 23: 87-95, 1995.
43. Wang, W., Liu, L., and Higuchi, C. Mucosal polyamine measurements and colorectal cancer risk. J. Cell. Biochem., 63: 252-257, 1996.
44. Ernestus, R.-I., Rohn, G., Schroder, R., Els, T., Lee, J.-Y., Klug, N., and Paschen, W. Polyamine metabolism in gliomas. J. Neuro-Oncol., 29: 167-174, 1996.
45. Chanda, R. and Ganguly, A. Diamine-oxidase activity and tissue di- and poly-amine contents of human ovarian, cervical and endometrial carcinoma. Cancer Lett., 89: 23-28, 1995.
46. Konety, B. R. and Getzenberg, R. H. Novel therapies for advanced prostate cancer. Semin Urol Oncol, 15: 33-42., 1997.
47. Lee, S., Suh, J., Chung, B., and Kim, S. Polyamine profile in the urine of patients with leukemia. Cancer Lett., 122: 1-8, 1998.
48. Hiramatsu, K., Sugimoto, M., Kamei, S., Hoshino, M., Kinoshita, K., Iwasaki, K., and Kawakita, M. Diagnostic and prognostic usefulness of N1,N8-diacetylspermidine and N1,N12-diacetylspermine in urine as novel markers of malignancy. J. Cancer Res. Clin. Oncol., 123: 539-545, 1997.
49. Cipolla, B., Moulinoux, J.-P., Quemener, V., Havouis, R., Martin, L.-A., Guille, F., and Lobel, B. Erythrocyte polyamine levels in human prostatic carcinoma. J. Urol, 144: 1164-1166, 1990.
50. Cipolla, B. G., Ziade, J., Bansard, J. Y., Moulinoux, J. P., Staerman, F., Quemener, V., Lobel, B., and Guille, F. Pretherapeutic erythrocyte polyamine spermine levels discriminate high risk relapsing patients with M1 prostate carcinoma. Cancer, 78: 1055-1065., 1996.
51. Liu, X., Wang, L., Lin, Y., Teng, Q., Zhao, C., Hu, H., and Chi, W. Ornithine decarboxylase activity and its gene expression are increased in benign hyperplastic prostate. Prostate, 43: 83-87., 2000.
52. Seiler, N., Atanassov, C. L., and Raul, F. Polyamine metabolism as target for cancer chemoprevention (Review). International Journal of Oncology, 13: 993-1006, 1998.
53. Shanna, R. A., Manson, M. M., Gescher, A., and Steward, W. P. Colorectal cancer chemoprevention: biochemical targets and clinical development of promising agents. Eur J Cancer, 37: 12-22., 2001.
54. Janne, J., Poso, H., and Raina, A. Polyamines in rapid growth and cancer. Biochim. Biophys. Acta., 473: 241-293, 1978.
55. Coffino, P. Regulation of cellular polyamines by antizyme. Nat Rev Mol Cell Biol, 2: 188-194., 2001.
56. Lovaas, E. Hypothesis: spermine may be an important epidermal antioxidant. Med Hypotheses, 45: 59-67., 1995.
57. Ha, H. C., Sirisoma, N. S., Kuppusamny, P., Zweier, J. L., Woster, P. M., and Casero, R. A., Jr. The natural polyamine spermidine functions directly as a free radical scavenger. Proc Natl Acad Sci USA, 95: 11140-11145., 1998.
58. Seiler, N., Delcros, J., and Moulinoux, J. Polyamine transport in mammalian cells. An update. Int. J. Biochem. Cell Biol., 28: 843-861, 1996.
59. Grillo, M. and Colombatto, S. Polyamine transport in cells. Biochem. Soc. Trans., 22: 894-898, 1994.
60. Thomas, T. and Thomas, T. J. Polyamines in cell growth and cell death: molecular mechanisms and therapeutic applications. Cell Mol Life Sci, 58: 244-258., 2001.
61. Muskiet, F. A., Dorhout, B., van den Berg, G. A., and Hessels, J. Investigation of polyamine metabolism by high-perfolmance liquid chromatographic and gas chromatographic profiling methods. J Chlomatogr B Biomed Appl, 667: 189-198., 1995.
62. Hunter, K. J. and Fairlamb, A. H. The determination of polyamines and amino acids by a fluorescamine-HPLC method. Methods Mol Biol, 79: 125-130., 1998.
63. Madhubala, R. Thin-layer chromatographic method for assaying polyamines. Methods Mol Biol, 79: 131-136., 1998.
64. Medina, M. A., Quesada, A. R., Nunez de Castro, I., and Sanchez-Jimenez, F. Histamine, polyamines, and cancer. Biochem Pharmacol, 57: 1341-1344., 1999.
65. Koike, C., Chao, D. T., and Zetter, B. R. Sensitivity to polyamine-induced growth arrest correlates with antizyme induction in prostate carcinoma cells. Cancer Res, 59: 6109-6112., 1999.
66. Harrison, G. Spermine in human testis. Biochem. J, 25: 1885-1892, 1931.
67. Simone, N. L., Remaley, A. T., Charboneau, L., Petricoin, E. F., 3rd, Glickman, J. W., Emmert-Buck, M. R., Fleisher, T. A., and Liotta, L. A. Sensitive immunoassay of tissue cell proteins procured by laser capture microdissection. Am J Pathol, 156: 445-452., 2000.
68. Paweletz, C. P., Liotta, L. A., and Petricoin, E. F., 3rd New technologies for biomarker analysis of prostate cancer progression: Laser capture microdissection and tissue proteomics. Urology, 57: 160-163., 2001.
69. Urenjak, J., Williams, S. R., Gadian, D. G., and Noble, M. Proton nuclear magnetic resonance spectroscopy unambiguously identifies different neural cell types. J. Neurosci., 13: 981-989, 1993.
70. Straubinger, K., Schick, F., and Lutz, O. Influence of pulse angle variations on stimulated echo acquisition mode proton nuclear magnetic resonance spectra of AB spin systems: theory and experiments with citrate. Magnetic Resonance Materials in Physics, Biology and Medicine, 7: 88-94, 1998.
71. Cox, I. Development and applications of in vivo clinical magnetic resonance spectroscopy. Prog Biophys Mol Biol, 65: 45-81, 1996.
72. Wiechert, W. and de Graaf, A. In vivo stationary flux analysis by 13C labeling experiments. Adv Biochem Eng Biotechnol, 54: 109-154, 1996.
73. Parvivar, F., Hricak, H., Shinohara, K., Kurhanewicz, J., Vigneron, D., Nelson, S., and Carroll, P. Detection of locally recurrent prostate cancer after cryosurgery: evaluation by transrectal ultrasound, magnetic resonance imaging, and three-dimensional proton magnetic resonance spectroscopy. Urology, 48: 594-599, 1996.
74. Kurhanewicz, J., Vigneron, D., Hricak, H., Parivar, F., Nelson, S., Shinohara, K., and Carroll, P. Prostate cancer: metabolic response to cryosurgery as detected with 3D H-1 MR spectroscopic imaging. Radiology, 200: 489-496, 1996.
75. Heerschap, A., Jager, G., van der Graaf, M., Barentsz, J., de la Rosette, J., Oosterhof, G., Ruijter, E., and Ruijs, S. In vivo proton MR spectroscopy reveals altered metabolite content in malignant prostate tissue. Anticancer Res, 17: 1455-1460, 1997.
76. Heerschap, A., Jager, G., van der Graaf, M., Barentsz, J., and Ruijs, S. Proton MR spectroscopy of the normal human prostate with an endorectal coil and a double spin-echo pulse sequence. Magn Reson Med, 37: 204-213, 1997.
77. van der Graaf, M., van den Boogert, H., Jager, G., Barentsz, J., and Heerschap, A. Human prostate: Multisection proton MR spectroscopic imaging with a single spin-echo sequence—preliminary experience. Radiology, 213: 919-925, 1999.
78. Costello, L., Franklin, R., and Narayan, P. Citrate in the diagnosis of prostate cancer. Prostate, 38: 237-245, 1999.
79. Swanson, M. G., Vigneron, D. B., Tran, T. K., Sailasuta, N., Hurd, R. E., and Kurhanewicz, J. Single-voxel oversampled J-resolved spectroscopy of in vivo human prostate tissue. Magn Reson Med, 45: 973-980., 2001.
80. Yu, K. K., Scheidler, J., Hricak, H., Vigneron, D. B., Zaloudek, C. J., Males, R. G., Nelson, S. J., Carroll, P. R., and Kurhanewicz, J. Prostate cancer: prediction of extracapsular extension with endorectal MR imaging and three-dimensional proton MR spectroscopic imaging. Radiology, 213: 481-488., 1999.
81. Kurhanewicz, J., Swanson, M. G., Wood, P. J., and Vigneron, D. B. Magnetic resonance imaging and spectroscopic imaging: Improved patient selection and potential for metabolic intermediate endpoints in prostate cancer chemoprevention trials. Urology, 57: 124-128., 2001.
82. Lynch, M., Masters, J., Pryor, J., Lindon, J., Spraul, M., Foxall, P., and Nicholson, J. Ultra high field NMR spectroscopic studies on human seminal fluid, seminal vesicle and prostatic secretions. J Pharm Biomed Anal, 12: 5-19, 1994.
83. Lynch, M. and Nicholson, J. Proton MRS of human prostatic fluid: correlations between citrate, spermine, and myo-inositol levels and changes with disease. Prostate, 30: 248-255, 1997.

84. Willker, W., Flogel, U., and Leibfritz, D. A 1H/13C inverse 2D method for the analysis of the polyamines putrescine, spermidine ans spermine in cell extracts and biofluids. NMR Biomed., 11: 47-54, 1998.
85. Halm, P., Smith, I., Leboldus, L., Littman, C., Somorjai, R., and Bezabeh, T. The classification of benign and malignant human prostate tissue by multivariate analysis of 1H magnetic resonance spectra. Cancer Res, 57: 3398-3401, 1997.
86. van der Graaf, M., Schipper, R., Oosterhof, G., Schalken, J., Verhofstad, A., and Heerschap, A. Proton MR spectroscopy of prostatic tissue focused on the detection of spermine, a possible biomarker of malignant behavior in prostate cancer. MAGMA, 10: 153-159, 2000.
87. Cornel, E., Smits, G., Oosterhof, G., Karthaus, H., Deburyne, F., Schalken, J., and Heerschap, A. Characterization of human prostate cancer, benign prostatic hyperplasia and normal prostate by in vitro 1H and 31P magnetic resonance spectroscopy. J Urol, 150: 2019-2024, 1993.
88. Kurhanewicz, J., Dahiya, R., Macdonald, J., Chang, L., James, T., and Narayan, P. Citrate alterations in primary and metastatic human prostatic adenocarcinomas: 1H magnetic resonance spectroscopy and biochemical study. Magn Reson Med, 29: 149-157, 1993.
89. Fowler, A., Pappas, A., Holder, J., Finkbeiner, A., Dalrymple, G., Mullins, M., Sprigg, J., and Komoroski, R. Differentiation of human prostate cancer from benign hypertrophy by in vitro 1H NMR. Magn Reson Med, 25: 140-147, 1992.
90. Schiebler, M., Miyamoto, K., White, M., Maygarden, S., and Mohler, J. In vitro high resolution 1H-spectroscopy of the human prostate: benign prostatic hyperplasia, normal peripheral zone and adenocarcinoma. Magn Reson Med, 29: 285-291, 1993.
91. Cornel, E., Smits, G., de Ruijter, J., Oosterhof, G., Heerschap, A., Debruyne, F., and Sclhalken, J. In vitro proton magnetic resonance spectroscopy of four human prostate cancer cell lines. Prostate, 26: 275-280, 1995.
92. Cheng, L. L., Ma, M. J., Becerra, L., Ptak, T., Tracey, I., Lackner, A., and Gonzalez, R. G. Quantitative neuropathology by high resolution magic angle spinning proton magnetic resonance spectroscopy. Proc Natl Acad Sci USA, 94: 6408-6413., 1997.
93. Cheng, L. L., Lean, C. L., Bogdanova, A., Wright, S. C., Jr., Ackerman, J. L., Brady, T. J., and Gamido, L. Enhanced resolution of proton NMR spectra of malignant lymph nodes using magic-angle spinning. Magn Reson Med, 36: 653-658., 1996.
94. Cheng, L. L., Chang, I. W., Smith, B., and Gonzalez, R. G. Evaluating human breast ductal carcinomas with high-resolution magic-angle-spinning proton magnetic resonance spectroscopy. J. Magn. Reson., 135: 194-202, 1998.
95. Cheng, L. L., Chang, I. W., Louis, D. N., and Gonzalez, R. G. Correlation of high-resolution magic angle spinning proton magnetic resonance spectroscopy with histopathology of intact human brain tumor specimens. Cancer Res, 58: 1825-1832., 1998.
96. Cheng, L. L., Anthony, D. C., Comite, A. R., Black, P. M., Tzika, A. A., and Gonzalez, R. G. Quantification of microheterogeneity in glioblastoma multiforme with ex vivo high-resolution magic-angle spinning (HRMAS) proton magnetic resonance spectroscopy. Neuro-oncol, 2: 87-95., 2000.
97. Cheng, L. L., Wu, C., Smith, M. R., and Gonzalez, R. G. Non-destructive quantitation of spermine in human prostate tissue samples using HRMAS 1H NMR spectroscopy at 9.4 T. FEBS Lett, 494: 112-116., 2001.
98. Barton, S. J., Howe, F. A., Tomlins, A. M., Cudlip, S. A., Nicholson, J. K., Bell, B. A., and Griffiths, J. R. Comparison of in vivo 1H MRS of huinan brain tumours with 1H HR-MAS spectroscopy of intact biopsy samples in vitro. Magma, 8: 121-128., 1999.
99. Bollard, M. E., Garrod, S., Holmes, E., Lindon, J. C., Humpfer, E., Spraul, M., and Nicholson, J. K. High-resolution (1)H and (1)H-(13)C magic angle spinning NMR spectroscopy of rat liver. Magn Reson Med, 44: 201-207., 2000.
100. Garrod, S., Humpfer, E., Spraul, M., Connor, S. C., Polley, S., Connelly, J., Lindon, J. C., Nicholson, J. K., and Holmes, E. High-resolution magic angle spinning 1H NMR spectroscopic studies on intact rat renal cortex and medulla. Magn Reson Med, 41: 1108-1118., 1999.
101. Garrod, S., Humpher, E., Connor, S. C., Connelly, J. C., Spraul, M., Nicholson, J. K., and Holmes, E. High-resolution (1)H NMR and magic angle spinning NMR spectroscopic investigation of the biochemical effects of 2-bromoethanamine in intact renal and hepatic tissue. Magn Reson Med, 45: 781-790., 2001.
102. Griffin, J. L., Troke, J., Walker, L. A., Shore, R. F., Lindon, J. C., and Nicholson, J. K. The biochemical profile of rat testicular tissue as measured by magic angle spinning 1H NMR spectroscopy. FEBS Lett, 486: 225-229., 2000.
103. Griffin, J. L., Walker, L. A., Garrod, S., Holmes, E., Shore, R. F., and Nicholson, J. K. NMR spectroscopy based metabonomic studies on the comparative biochemistry of the kidney and urine of the bank vole (Clethrionomys glareolus), wood mouse (Apodemus sylvaticus), white toothed shrew (Crocidura suaveolens) and the laboratory rat. Comp Biochem Physiol B, 127: 357-367., 2000.
104. Griffin, J. L., Walker, L. A., Troke, J., Osborn, D., Shore, R. F., and Nicholson, J. K. The initial pathogenesis of cadmium induced renal toxicity. FEBS Lett, 478: 147-150., 2000.
105. Humpfer, E., Spraul, M., Nicholls, A. W., Nicholson, J. K., and Lindon, J. C. Direct observation of resolved intracellular and extracellular water signals in intact human red blood cells using 1H MAS NMR spectroscopy. Magn Reson Med, 38: 334-336., 1997.
106. Millis, K. K., Maas, W. E., Cory, D. G., and Singer, S. Gradient, high-resolution, magic-angle spinning nuclear magnetic resonance spectroscopy of human adipocyte tissue. Magn Reson Med, 38: 399-403., 1997.
107. Millis, K., Weybright, P., Campbell, N., Fletcher, J. A., Fletcher, C. D., Cory, D. G., and Singer, S. Classification of human liposarcoma and lipoma using ex vivo proton NMR spectroscopy. Magn Reson Med, 41: 257-267., 1999.
108. Moka, D., Vorreuther, R., Schicha, H., Spraul, M., Humpfer, E., Lipinski, M., Foxall, P. J., Nicholson, J. K., and Lindon, J. C. Biochemical classification of kidney carcinoma biopsy samples using magic-angle-spinning 1H nuclear magnetic resonance spectroscopy. J Pharm Biomed Anal, 17: 125-132., 1998.
109. Tate, A. R., Foxall, P. J., Holmes, E., Moka, D., Spraul, M., Nicholson, J. K., and Lindon, J. C. Distinction between normal and renal cell carcinoma kidney cortical biopsy samples using pattern recognition of (1)H magic angle spinning (MAS) NMR spectra. NMR Biomed, 13: 64-71., 2000.
110. Waters, N. J., Garrod, S., Fanant, R. D., Haselden, J. N., Connor, S. C., Connelly, J., Lindon, J. C., Holmes, E., and Nicholson, J. K. High-resolution magic angle spinning (1)H NMR spectroscopy of intact liver and kidney: optimization of sample preparation procedures and biochemical stability of tissue during spectral acquisition. Anal Biochem, 282: 16-23., 2000.
111. Weybright, P., Millis, K., Campbell, N., Cory, D. G., and Singer, S. Gradient, high-resolution, magic angle spinning 1H nuclear magnetic resonance spectroscopy of intact cells. Magn Reson Med, 39: 337-345., 1998.
112. Gonzalez, R. G., Cheng, L. L., Westmoreland, S. V., Sakaie, K. E., Becerra, L. R., Lee, P. L., Masliah, E., and Lackner, A. A. Early brain injury in the SIV-macaque model of AIDS. Aids, 14: 2841-2849., 2000.
113. Ruiz-Cabello, J. and Cohen, J. S. Phospholipid Metabolites as Indicators of Cancer Cell Function. NMR in Biomedicine, 5: 226-233, 1992.
114. Podo, F. and de Certaines, J. Magnetic resonance spectroscopy in cancer: phospholipid, neutral lipid and lipoprotein metabolites and function. Anticancer res, 16: 1305-1316, 1996.
115. Hakumaki, J. M. and Kauppinen, R. A. 1H NMR visible lipids in the life and death of cells. Trends Biochem Sci, 25: 357-362., 2000.
116. Blankenberg, F. G., Katsikis, P. D., Storrs, R. W., Beaulieu, C., Spielman, D., Chen, J. Y., Naumovski, L., and Tait, J. F. Quantitative analysis of apoptotic cell death using proton nuclear magnetic resonance spectroscopy. Blood, 89: 3778-3786., 1997.
117. Mountford, C., Lean, C., Hancock, R., Dowd, S., Mackinnon, W., Tattersall, M., and Russell, P. Magnetic resonance spectroscopy detects cancer in draining lymph nodes. Invasion & Metastasis, 13: 57-71, 1993.
118. Mountford, C., Lean, C. L., Mackinnon, W., and Russell, P. The use of proton MR in cancer pathology. In: G. Webb (ed.), Annual report on NMR spectroscopy, Vol. 27, pp. 173-215. London: Academic Press, 1993.
119. Gillies, R. J., Bbujwalla, Z. M., Eve/loch, J., Garwood, M., Neeman, M., Robinson, S. P., Sotak, C. H., and Van Der Sanden, B. Applications of magnetic resonance in model systems: tumor biology and physiology. Neoplasia, 2: 139-151., 2000.
120. Evelhoch, J. L., Gillies, R. J., Karczmar, G. S., Koutcher, J. A., Maxwell, R. J., Nalcioglu, O., Raghunand, N., Ronen, S. M., Ross, B. D., and Swartz, H. M. Applications of magnetic resonance in model systems: cancer therapeutics. Neoplasia, 2: 152-165., 2000.
121. Hyttinen, I. M., Halmekyto, M., Alhonen, L., and Janne, J. Levels of ornithine decarboxylase genomic sequences, heterogeneous nuclear RNA and mRNA in human myeloma cells resistant to alpha-difluoromethylornithine. Biochem J, 278: 871-874., 1991.
122. Wind, R. A., Hu, J. Z., and Rommereim, D. N. High-resolution (1)H NMR spectroscopy in organs and tissues using slow magic angle spinning. Magn Reson Med, 46: 213-218., 2001.
123. Song, Z., Antzutkin, O. N., Feng, X., and Levitt, M. H. Sideband suppression in magic-angle-spinning NMR by a sequence of 5 pi pulses. Solid State Nucl Magn Reson, 2: 143-146., 1993.
124. Petroff, O., Ogino, T., and Alger, J. High-resolution proton magnetic resonance spectroscopy of rabbit brain: regional metabolites levels and postmortem changes. J Neurochem, 51: 163-171, 1988.
125. Lowry, M., Liney, G., Turnbull, L., Manton, D., Blackband, S., and Horsman, A. Quantification of citrate concentration in the prostate by proton magnetic resonance spectroscopy: zonal and age-related differences. Magn Reson Med, 36: 352-358, 1996.
126. Liney, G., Lowry, M., Turnbull, L., Manton, D., Knowles, A., Blackband, S., and Horsman, A. Proton MR T2 maps correlate with the citrate concentration in the prostate. NMR Biomed, 9: 59-64, 1996.
127. Liney, G., Turnbull, L., Lowry, M., Turnbull, L., Knowles, A., and Horsman, A. In vivo quantification of citrate concentration and water T2 relaxation time of the pathologic prostate gland using 1H MRS and MRI. Magn Reson Imaging, 15: 1177-1186, 1997.
128. Hendrick, R. Issues in quantitation of MR parameters for breast cancer. Proc. ISMRM, 4: 334, 1996.
129. Wang, Y., Xiao, L., Thiagalingam, A., Nelkin, B. D., and Casero, R. A., Jr. The identification of a cis-element and a trans-acting factor involved in the response to polyamines and polyamine analogues in the regulation of the human spermidine/spermine N1-acetyltransferase gene transcription. J Biol Chem, 273: 34623-34630., 1998.
130. Manni, A., Grove, R., Kunselman, S., and Aldaz, M. Involvement of the polyamine pathway in breast cancer progression. Cancer Lett., 92: 49-57, 1995.
131. Kauppinen, L., Myohanen, S., Halmekyto, M., Alhonen, L., and Janne, J. Transgenic mice over-expressing the human spermidine synthase gene. Biochem J, 293: 513-516., 1993.
132. Green, M. L., Blaeser, L. L., Simmen, F. A., and Simmen, R. C. Molecular cloning of spermidine/spermine N1-acetyltransferase from the periimplantation porcine uterus by messenger ribonucleic acid differential display: temporal and conceptus-modulated gene expression. Endocrinology, 137: 5447-5455., 1996.
133. Korhonen, V. P., Halmekyto, M., Kauppinen, L., Myohanen, S., Wahlfors, J., Keinanen, T., Hyvonen, T., Albonen, L., Eloranta, T., and Janne, J. Molecular cloning of a cDNA encoding human spermine synthase. DNA Cell Biol, 14: 841-847., 1995.
134. Cox, D. R. Regression models and life tables. J R Stat Soc [Ser B], 34: 187-220, 1972.
135. Harris, E. K. and Albert, A. Survivorship analysis for clinical studies. New York: Marcel Dekker, 1991.
136. Lee, E. T. Statistical methods for survival data analysis. New York: John Wiley & Sons, 1992.
137. Bax, A., Mehlkopf, A., and Smidt, J. J. Magn. Reson, 35: 167, 1979.
138. Maudsley, A. A. and Ernst, R. Chem Phys Lett, 50: 368, 1977.
139. Bak, M., Thomsen, J. K., Jakobsen, H. J., Petersen, S. E., Petersen, T. E., and Nielsen, N. C. Solid-state 13C and 31P NMR analysis of urinary stones. J Urol, 164: 856-863., 2000.
140. Guo, W., Monisett, J. D., DeBakey, M. E., Lawrie, G. M., and Hamilton, J. A. Quantification in situ of crystalline cholesterol and calcium phosphate hydroxyapatite in human atherosclerotic plaques by solid-state magic angle spinning NMR. Arterioscler Thromb Vasc Biol, 20: 1630-1636., 2000.
141. Wind, R. A., Concannon, B. A., Groch, K. M., Rommereim, D. N., and Santos, R. A. An investigation of rat mammary healthy and R3230AC tumor tissues and cells by means of solid-state 13C NMR. Solid State Nucl Magn Reson, 7: 263-269., 1996.

The references cited throughout the application are incorporated herein by reference in their entirety.

What is claimed is:

1. A method for prognostic evaluation of an individual suspected of having, or having, cancer of epithelial origin comprising:
a. isolating histologically-benign epithelia from a tissue sample comprising the cancer; and
b. measuring the level of mRNA in the epithelia corresponding to an enzyme in the spermine pathway, wherein an elevated level of mRNA indicates that the cancer in likely aggressive and therefore the individual has a poor prognosis.

2. The method of claim 1, wherein the histologically-benign epithelia is isolated using laser capture microdissection.

3. The method of claim 1, wherein the enzyme is selected from the group consisting of ornithine decarboxylase, spermidine/spermine N-acetyltransferase, spermidine synthase, spermidine synthase, spermine oxidase, and polyamine oxidase.

4. The method of claim 1, wherein the cancer is selected from the group consisting of breast cancer, basal cell carcinoma, adenocarcinoma, gastrointestinal cancer, lip cancer, mouth cancer, esophageal cancer, small bowel cancer, stomach cancer, colon cancer, liver cancer, bladder cancer, pancreas cancer, ovary cancer, cervical cancer, lung cancer, skin cancer, prostate cancer, and renal cell carcinoma.

5. The method of claim 1, wherein the cancer is prostate cancer.

6. A method for prognostic evaluation of an individual suspected of having, or having, prostate cancer comprising:
a. isolating histologically-benign prostate epithelia from a prostate biopsy; and
b. measuring the level of mRNA in the epithelia corresponding to an enzyme in the spermine pathway, wherein an elevated level of mRNA indicates that the prostate cancer in likely aggressive and therefore the individual has a poor prognosis.

7. The method of claim 6, wherein the histologically-benign prostate epithelia is isolated using laser capture microdissection.

8. The method of claim 6, wherein the enzyme is selected from the group consisting of ornithine decarboxylase, spermidine/spermine N-acetyltransferase, spermidine synthase, spermidine synthase, spermine oxidase, and polyamine oxidase.

9. A method for prognostic evaluation in an individual having cancer comprising measuring spermine pathway enzyme mRNA levels in histologically-benign epithelia of a tissue biopsy comprising the cancer, wherein an elevated level of enzyme mRNA indicates that the cancer is likely aggressive and the individual has a poor prognosis.

10. The method of claim 9, wherein the histologically-benign epithelia is isolated using laser capture microdissection.

11. The method of claim 9, wherein the spermine pathway enzyme is selected from the group consisting of ornithine decarboxylase, spermidine/spermine N-acetyltransferase, spermidine synthase, spermine synthase, spermine oxidase, and polyamine oxidase.

12. The method of claim 9, wherein the cancer is selected from the group consisting of breast cancer, basal cell carcinoma, adenocarcinoma, gastrointestinal cancer, lip cancer, mouth cancer, esophageal cancer, small bowel cancer, stomach cancer, colon cancer, liver cancer, bladder cancer, pancreas cancer, ovary cancer, cervical cancer, lung cancer, skin cancer, prostate cancer, and renal cell carcinoma.

13. The method of claim 9, wherein the cancer is prostate cancer.
